(19) United States
(12) Patent Application Publication
(10) Pub. No.: US 2006/0020164 A1
(43) Pub. Date: Jan. 26, 2006

(54) Evertable insertion tube for colonoscope

(75) Inventors: John Butler, Blackrock (IE); Frank Bonadio, Bray (IE); Shane Joseph MacNally, Bray (IE); Lisa Woods, Wicklow Town (IE); Duncan Bell, Falkenham Ipswich (GB)

Correspondence Address: KUDIRKA & JOBSE, LLP, ONE STATE STREET, SUITE 800, BOSTON, MA, 02109, US

(73) Assignee: Atropos Limited, Bray (IE)

(21) Appl. No.: 11/050,665

(22) Filed: Feb. 3, 2005

Related U.S. Application Data
(63) Continuation of application No. PCT/IE03/00110, filed on Aug. 6, 2003.

(30) Foreign Application Priority Data
Aug. 6, 2002 (IE) 2002/0658
Aug. 6, 2002 (IE) 2002/0656

(51) Int. Cl. A61B 1/00

(52) U.S. Cl. 600/115; 600/114; 600/159

(57) ABSTRACT

An evertable tube having a lumen therethrough, an inflation port for inflating the tube, and a self-closing valve at the inflation port. The tube has an inflation port for inflating the tube to grip a member, such as a probe, in the lumen. Advancement of the member, for example through a colon, will then cause eversion of the tube. The inflation port is provided in the form of an aperture in the outer wall of the tube. The valve comprises a sheath extending from the port along a wall of the tube. The sheath may be fixed to the wall of the tube. The sheath may alternatively be integrally formed with the wall of the tube. The sheath extends from the inflation port along the wall of the tube parallel to the longitudinal axis of the tube to an open end of the sheath.

### RELATED APPLICATIONS

This application is a continuation application of PCT application PCT/IE2003/000110, filed Aug. 6, 2003 and which claimed priority to Ireland Patent Application Nos. 2002/0658 and 2002/0656 both filed Aug. 6, 2002, the contents of all which are incorporated herein by reference.

### FIELD OF THE INVENTION

This invention relates to a guide device for assisting advancement of a probe through a passageway by maintaining the probe spaced from the interior walls of the passageway during advancement of the probe through the passageway. In particular this invention relates to a guide device which facilitates enhanced vision during probe advancement, especially in a tortuous passageway such as the colon.

### BACKGROUND OF THE INVENTION

Conventional colonoscopy procedures involve advancing a colonoscope through the floppy sigmoid colon to the proximal end of the descending colon.

However, advancing a colonoscope through the sigmoid colon generally causes loops to form in the floppy sigmoid colon, and stretches the mesentery to which the sigmoid colon is attached. This results in considerable pain and discomfort for the patient.

It is known to use an everting tube to advance a probe through a passageway. For example, U.S. Pat. No. 4,321,915 describes such a flexible, everting tube. By applying a fluid pressure to the tube, a fibre optic tool extending through the tube is gripped and pulled along by the tube as it everts. One problem with known guide devices of this type is that because of the tube eversion action, a tool extending through the tube advances at twice the rate of the tube. Thus, the tip of the tool extends beyond the leading edge of the everting tube. U.S. Pat. No. 4,321,915 describes applying a suction to the tube when the tip of the tool has extended a distance beyond the leading edge of the tube. The suction causes the tube to disengage from the tool and allows an operator to manually retract the tool into the tube.

This procedure is generally inconvenient and inefficient, especially when navigating tortuous passageways such as the colon.

The lower gastrointestinal tract comprises the rectum, and the large intestine or colon. The colon, in a textbook arrangement of the human anatomy, extends upwards from the lower right quadrant, traverses the width of the body just below the diaphragm, travels downwards along the left side of the abdomen and then loops in an anterior retrograde manner before linking up with the rectum and the anus.

Even in such a textbook arrangement, the large intestine is difficult to cannulate with a colonoscope due to the flexible nature of the colonoscope and the floppy nature of the colon. This is even more difficult with the more realistic anatomies of actual people.

In some people, the sigmoid colon can be very long and is unfixed, except by its mesentery, and so can be extremely difficult to cannulate due to its predisposition to form loops when a colonoscope is pushed through it. Looping of the colonoscope within the sigmoid colon and transverse colon exacerbates the problems in traversing these areas.

Conventional colonoscopy procedures involve advancing a colonoscope through the floppy sigmoid colon to the proximal end of the descending colon. During advancement of the colonoscope through the sigmoid colon loops often form. It is difficult to then advance the colonoscope further, due to the looped nature of the sigmoid colon. Further pushing of the colonoscope simply increases the loops in the sigmoid colon without advancing the colonoscope into the descending colon.

The sigmoid colon is generally straightened by manipulation of the colonoscope. However advancing the colonoscope further, into the descending colon may cause the loops in the floppy sigmoid colon to reform.

It is known to use an overtube to prevent the reformation of loops by splinting the straightened sigmoid colon. The overtube is typically advanced over the colonoscope until the distal end of the overtube is at the proximal end of the descending colon. The overtube then maintains the sigmoid colon in the straightened configuration and prevents loops from reforming in the sigmoid colon during advancement of the colonoscope further, into the descending colon.

However, due to the potentially tortuous path through a colon, it is often difficult to advance an overtube over a colonoscope without kinking of the overtube occurring.

Furthermore, parts of the interior wall of a colon may become trapped between a colonoscope and an overtube during advancement of the overtube over the colonoscope. This may result in shearing off of the trapped part of the colon wall or puncturing of the colon wall.

In addition, in certain colonoscopy procedures, for example a multiple polypectomy, it is necessary to insert and remove a colonoscope several times. This requires considerable skill on the part of the colonoscopist and takes a considerable length of time.

### SUMMARY OF THE INVENTION

According to the invention there is provided an evertable tube having a lumen therethrough, an inflation port for inflating the tube, and a self-closing valve at the inflation port.

In one embodiment of the invention, the valve comprises a sheath extending from the port along a wall of the tube.

The sheath may extend substantially parallel to the longitudinal axis of the tube.

In one case, the sheath is fixed to the wall of the tube. In one embodiment, the sheath is integral with the wall of the tube. The sheath may be of the same material as the wall of the tube.

In another embodiment, the tube has two or more inflation ports. The tube may be a self-closing valve at each inflation port.

The tube may be at least partially twisted. In one case, the tube comprises means for adjusting the twist in the tube. The tube comprises a substantially cylindrical outer sleeve section and a twisted inner sleeve section, the inner sleeve section being of the same untwisted diameter as that of the outer sleeve section.

At least a portion of the tube may have a non-linear shape. The tube may be biased into the non-linear shape. The tube may be sculpted or formed into the non-linear shape.

In another aspect, the invention provides a guide device for a probe, the device comprising: an evertable tube with a lumen therethrough, the tube being inflatable to grip a probe in the lumen such that the tube everts with advancement of the probe; and means to facilitate pulling a probe back relative to the tube to realign the probe relative to the tube.

In one embodiment, the device comprises means to longitudinally stiffen the tube. The stiffening means may comprise at least one bracing column located within the tube.

In a further aspect, the invention provides a guide device for a probe, the device comprising: an evertable tube with a lumen therethrough, the tube being inflatable to grip a probe in the lumen such that the tube everts with advancement of the probe; and at least one stiffening column to facilitate moving at least part of the tube distally relative to a probe in the lumen to align an end of the tube with an end of the probe.

The column may be tubular and extend co-axially around the lumen.

The column may comprise at least one corrugation for kink resistance and the corrugation may extend along the column in a convoluted manner.

The corrugation may extend around the column in a loop.

In another case, the column has at least one slit through the column wall extending along the column in a spiral.

The outer wall of the tube may be connected at each end to the inner wall of the tube to define an enclosed inflation space therebetween.

In one case the tube comprises an evertable tube of the invention.

The device may comprise stop means to selectively prevent tube eversion. The stop means may comprise a clamp engageable with the tube.

The invention provides in one case a guide device for a colonoscope.

According to another aspect of the invention, there is provided a probe assembly comprising: a probe; and a guide device for the probe, the device comprising: an evertable tube with a lumen therethrough, the tube being inflatable to grip a probe in the lumen such that the tube everts with advancement of the probe.

In one case, the probe comprises means to create a fluid cushion between the probe and the guide device. The probe may comprise one or more fluid openings on an outer surface of the probe for ejecting fluid therethrough to create the fluid cushion. The probe may also comprise a fluid inlet lumen in communication with the openings for passage of a fluid through the lumen and out through the openings. The probe may comprise a fluid exhaust lumen for passage of a fluid through the lumen.

The tube may comprise an evertable tube.

The device may comprise a guide device.

In one case the probe comprises a colonoscope.

In another aspect of the invention, there is provided a device for straightening a looped section of a passageway, the device comprising: an anchor for anchoring an interior wall of a passageway to the anchor; and means for advancing the anchor through a passageway.

In one embodiment, the anchor is movable outwardly to anchor an interior wall of a passageway to the anchor by exerting outward pressure on the interior wall of the passageway. At least part of the anchor may be inflatable.

The anchor may comprise an evertable tube. In one case, the tube comprises an evertable tube.

The anchor may comprise a guide device.

In one case, the means for advancing the anchor comprises a probe.

The device may comprise a probe assembly.

In one case, the device is suitable for straightening a sigmoid colon.

According to a further aspect, the invention provides a method of advancing a probe through a passageway, the method comprising: providing a probe;
providing a guide device for the probe, the guide device having a lumen therethrough;
inserting the probe through the lumen of the guide device; inflating the guide device to grip the probe; advancing the probe through the passageway with an associated eversion of the guide device; and retracting the probe relative to the guide device to align the leading end of the guide device with the leading end of the probe.

The guide device may be advanced over the probe by pushing the guide device over the probe.

The method may comprise releasing a stop means before advancing at least part of the guide device over the probe.

The probe may be maintained in a fixed position during the step of advancing at least part of the guide device over the probe.

The probe may be advanced through the passageway by pushing the probe through the passageway.

In a further aspect, the invention provides a method of advancing a probe through a passageway, the method comprising the steps of: providing a probe; providing a guide device for the probe, the guide device having a lumen therethrough; inserting the probe through the lumen of the guide device; inflating the guide device to grip the probe; pushing the probe to advance the probe through the passageway which causes eversion of the guide device; retracting the probe relative to the guide device to align the leading end of the guide device with the leading end of the probe.

In one case, at least part of the guide device is advanced over the probe to align the leading end of the guide device with the leading end of the probe.

The probe may be retracted through the lumen to align a leading end of the guide device with a leading end of the probe.

The method may comprise moving the guide device out of contact with the probe before retracting the probe relative to the guide device.

The invention provides, in another aspect, a method of advancing a probe through a passageway, the method comprising: providing a probe; providing a guide device for the probe, the guide device having a lumen therethrough; inserting the probe through the lumen of the guide device; inflating the guide device to grip the probe; advancing the probe through the passageway with an associated eversion of the guide device; moving the guide device out of contact with the probe; and moving at least part of the guide device distally relative to the probe to align the leading end of the guide device with the leading end of the probe.

In one case, the guide device is moved out of contact with the probe by deflating the guide device.

The guide device may be moved out of contact with the probe by creating a fluid cushion between the guide claim and the probe.

The method may comprise releasing a stop means before eversion of the guide device.

At least some of the steps may be repeated to advance the probe in an incremental manner through the passageway.

In one case, the method is suitable for advancing a colonoscope through a colon.

The invention provides, in a further aspect, a method of straightening a looped section of a passageway, the method comprising: providing an anchoring device; advancing the anchoring device through the passageway; anchoring the interior wall of the passageway to the anchoring device; and moving the anchoring device proximally to at least partially straighten the section of the passageway.

In one case, the anchoring device exerts outward pressure on the interior wall of the passageway to anchor the interior wall of the passageway to the anchoring device.

The interior wall of the passageway may be anchored to the anchoring device during advancement of the anchoring device through the passageway.

The anchoring device may evert during advancement through the passageway.

At least some of the steps may be repeated to straighten the looped section of the passageway in an incremental manner.

The method may be a method of straightening a sigmoid colon.

According to the invention, there is also provided a method of performing a colonoscopy procedure, the method comprising: inserting a colonoscope into a colon; inserting a colonic overtube into the colon; advancing the colonoscope through a floppy section of the colon; straightening the floppy section of the colon; advancing the colonic overtube over the colonoscope through the floppy section of the colon; and advancing the colonoscope distally of the straightened section of the colon.

In one embodiment of the invention, m the floppy section of the colon is straightened by manipulating the colonoscope and/or the colonic overtube from externally of the colon.

The colonic overtube may be advanced distally of the straightened section of the colon.

In another aspect, the invention provides a method of performing a colonoscopy procedure, the method comprising: inserting a colonoscope into a colon; inserting a colonic overtube into the colon; advancing the colonoscope through a floppy section of the colon; advancing the colonic overtube over the colonoscope through the floppy section of the colon; and advancing the colonoscope distally of the floppy section of the colon.

In one case, the colonic overtube is advanced distally of the floppy section of the colon.

The floppy section of the colon may comprise the transverse colon.

According to a further aspect of the invention, there is provided a method of performing a colonoscopy procedure, the method comprising: inserting a colonoscope into a colon; inserting a colonic overtube into the colon; advancing the colonoscope around a bend in the colon; advancing the colonic overtube over the colonoscope around the bend in the colon; and advancing the colonoscope distally of the bend in the colon.

In one embodiment, the colonic overtube remains in a fixed position during advancement of the colonoscope distally of the bend in the colon.

The bend may comprise the splenic flexure.

In another embodiment, the method comprises withdrawing the colonoscope from the colon while the colonic overtube remains in place in the colon. The method may comprise advancing a medical device through the colonic overtube to access a point in the colon distally of the colonic overtube.

The method may comprise mounting the colonic overtube to the colonoscope before inserting the colonoscope into the colon.

The colonic overtube may be advanced by extending at least part of the colonic overtube from a shortened configuration to an elongated configuration.

In another case the colonic overtube is advanced by pushing the colonic overtube from externally of the colon.

The laterally flexible nature of the colonic overtube enables the overtube to advance through a potentially tortuous path in a colon without kinking. This is particularly advantageous when the overtube is being advanced through a sharp bend in the colon, for example, when advancing the overtube through the splenic or hepatic flexures or through parts of the sigmoid colon.

The colonic overtube has a flexible seal at the distal end of the overtube. The seal ensures that no parts of the colon wall become trapped between the overtube and the colonoscope during advancement of the overtube over the colonoscope. This arrangement prevents shearing off of the trapped part of the colon wall or puncturing of the colon wall.

In some colonoscopy procedures, air or some other gas is used to insufflate the colon, for example, to blow a protruding piece of the wall of the colon laterally to clear a path for advancement of the overtube and/or the colonoscope further distally through the colon. A further advantage of the seal is that it prevents insufflation air from leaking proximally out of the colon between the colonoscope and overtube.

In addition, the flexible nature of the seal enables the seal to adapt to the size of the colonoscope to achieve an effective seal between the overtube and the colonoscope for a variety of differently sized colonoscopes.

Because the colonic overtube is of a thermally stable material, the stiffness of the overtube may be chosen to be sufficiently flexible for ease of insertion into a colon, and to remain sufficiently stiff within the colon to maintain a section of the colon, such as the sigmoid colon, in a straightened configuration.

The colonic overtube provides an ergonomic and easily workable means of cannulating the colon as far distally as the caecum, without requiring a long, awkward length of tubing externally of the colon.

The rounded tip at the distal end of the colonic overtube ensures that the overtube advances atraumatically through the colon. Any inadvertent contact between the distal end of the overtube and the interior wall of the colon will not result in damage or trauma to the colon.

The colonic overtube provides a bridge between the fixed rectum and the fixed descending colon over the floppy sigmoid colon, thus preventing loops from reforming in the sigmoid colon. Furthermore, the colonic overtube provides a bridge between the fixed descending colon and the fixed ascending colon over the floppy transverse colon, thus preventing loops from reforming in the transverse colon. By using the overtube of the invention, advancement of a colonoscope through a colon as far as the caecum may be achieved easier and quicker, and causes less discomfort to a patient.

For an overtube to successfully splint a straightened sigmoid colon, its stiffness must be above the minimum threshold of stiffness required to prevent sigmoid loops from re-forming as the colonoscope is passed through the colonoscope lumen, and advanced further into the colon.

However it is also desirable that the overtube is not overly stiff, as insertion of the overtube becomes more difficult due to friction as the stiffness increases. This is because a “straightened” sigmoid colon is never perfectly straight. Consequently it is almost impossible to introduce a completely rigid overtube over the colonoscope. Some degree of compliance is required by the overtube.

While an overtube measured at room temperature may appear stiff enough to successfully splint a straightened sigmoid colon, this may no longer be the case at body temperature. Known overtube materials show a dramatic drop in stiffness between ambient room temperature and body temperature. In order for an overtube made from such materials to splint the sigmoid colon, it will have to be made overly rigid, so that it is still above the minimum threshold of stiffness required to prevent sigmoid loops from re-forming at body temperature. This excess rigidity causes serious insertion difficulties due to friction. Alternatively, if an overtube made from such materials was made less stiff, it may be easier to insert, but may not be stiff enough at body temperature to successfully splint the straightened sigmoid colon.

The colonic overtube described herein is configured to be relatively thermally stable. In this way the overtube at room temperature (insertion temperature) is selected to be sufficiently compliant or floppy to be easily inserted into a colon over a colonoscope. There is then a minimal drop in stiffness between ambient room temperature and body temperature compared to other materials, so that at body temperature the overtube is above the minimum threshold of stiffness required to prevent sigmoid loops from reforming.

Two other features of the overtube aid the insertion process: (a) corrugations, which minimize frictional contact with the scope; and (b) extremely low friction PTFE material used in its construction.

### BRIEF DESCRIPTION OF THE DRAWINGS

The above and further advantages of the invention may be better understood by referring to the following description in conjunction with the accompanying drawings in which:

FIG. 1 is a perspective view of a guide device according to the invention in a deflated configuration;

FIG. 2 is a perspective view of the guide device of FIG. 1 in an inflated configuration;

FIG. 3 is a side, cross-sectional view of the guide device of FIG. 1 in the deflated configuration;

FIG. 4 is a side, cross-sectional view of the guide device of FIG. 1 in the inflated configuration;

FIGS. 5 to 8 are views similar to FIGS. 1 to 4 of a probe extending through a lumen of the guide device of FIG. 1;

FIG. 9 is a side, partially cross-sectional view of a stiffening column of the guide device of FIG. 1;

FIGS. 10 and 11 are side views of other stiffening columns;

FIGS. 12 to 20 are side, partially cross-sectional views illustrating advancement of the probe of FIGS. 5 to 8 through a passageway using the guide device of FIG. 1;

FIGS. 21 to 28 are perspective views illustrating schematically advancement of the probe of FIGS. 5 to 8 through a curved passageway using the guide device of FIG. 1;

FIGS. 29 to 33 are partially cross-sectional, side views illustrating advancement of another probe according to the invention through a passageway using the guide device of FIG. 1;

FIGS. 34 and 35 are partially cross-sectional, side views illustrating advancement of a further probe according to the invention through a passageway using the guide device of FIG. 1;

FIGS. 36 to 48 are schematic views illustrating straightening of a sigmoid colon using the probe of FIGS. 34 and 35 and the guide device of FIG. 1;

FIG. 49 is a perspective view of a vision system;

FIG. 50 is a perspective view of an evertable tube according to the invention;

FIG. 51 is a plan view of the tube of FIG. 50;

FIGS. 52 and 53 are partially cross-sectional, side views of the tube of FIG. 50 in use;

FIG. 54 is a plan view of another evertable tube according to the invention;

FIG. 55 is a perspective view of a colonic overtube;

FIG. 56 is a partially cross-sectional, side view of a distal end of the overtube of FIG. 55;

FIGS. 57 to 61 are partially cross-sectional, side views illustrating manufacture of the overtube of FIG. 56;

FIG. 62 is a schematic view illustrating lubrication of the overtube of FIGS. 55 and 56;

FIGS. 63 and 64 are perspective views of a colonoscope extending through the overtube of FIG. 55;

FIG. 65 is a schematic view of a colon;

FIGS. 66 to 75 are schematic views of the colonoscope and overtube of FIGS. 63 and 64 in use in the colon of FIG. 65;

FIG. 76 is a partially cross-sectional, side view of the colonoscope of FIG. 63 advancing through the overtube of FIG. 63;

FIG. 77 is an enlarged, partially cross-sectional, side view of part of the colonoscope and overtube of FIG. 76;

FIG. 78 is a partially cross-sectional, side view of the colonoscope of FIG. 77 advancing through another colonic overtube;

FIGS. 79 and 80 are partially cut-away, perspective views of other colonic overtubes;

FIG. 81 is a perspective view of the colonoscope and overtube of FIG. 63 with a limiting means mounted to the overtube;

FIGS. 82 and 83 are partially cross-sectional, side views of the colonoscope, overtube and limiting means of FIG. 81;

FIG. 84 is a partially cross-sectional, side view of another colonic overtube;

FIGS. 85 to 90 are schematic views illustrating a method of performing a colonoscopy according to the invention using another colonic overtube;

FIG. 91 is a schematic view illustrating another method of performing a colonoscopy according to the invention using the overtube of FIGS. 85 to 90; and

FIG. 92 is a perspective view of a distal end of a further colonic overtube.

### DETAILED DESCRIPTION

Referring to the drawings there is illustrated a guide device 1 according to the invention. The guide device 1 is suitable for assisting advancement of a probe 7, such as a colonoscope, through a passageway, such as a body cavity 11, for example the colon.

The device 1 comprises an evertable tube 3 with a central lumen 8 therethrough. The tube 3 can be inflated to grip the probe 7 in the lumen 8 such that the tube 3 will evert with advancement of the probe 7. The device 1 further comprises means to facilitate moving at least part of the probe 7 through the tube 3 in the lumen 8 to align an end of the tube 3 with an end of the probe 7.

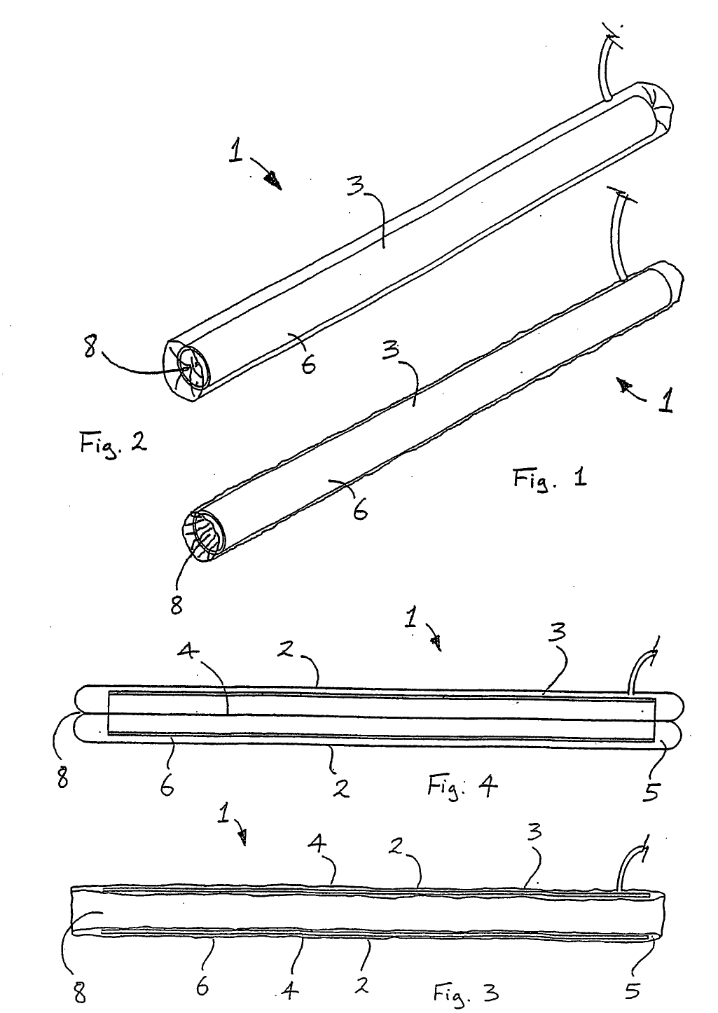

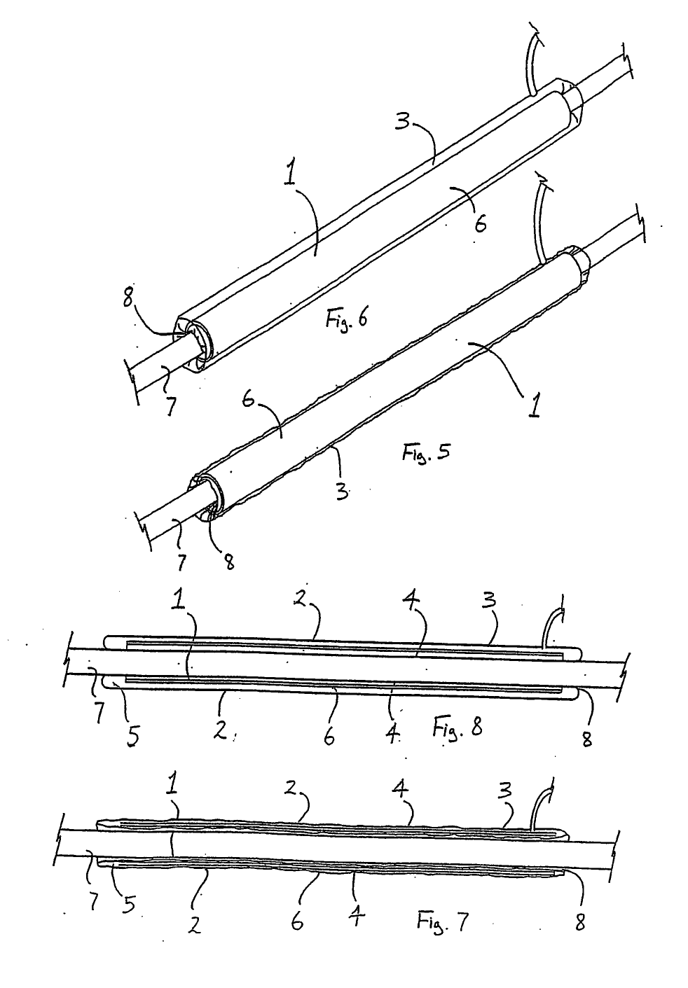

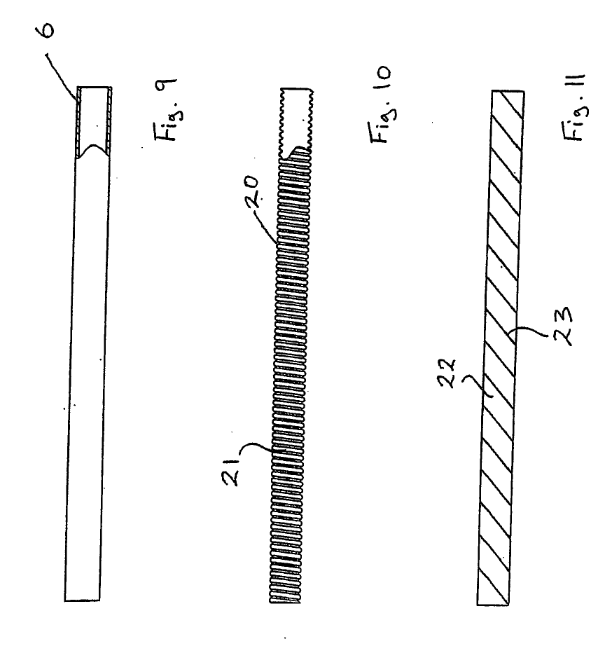

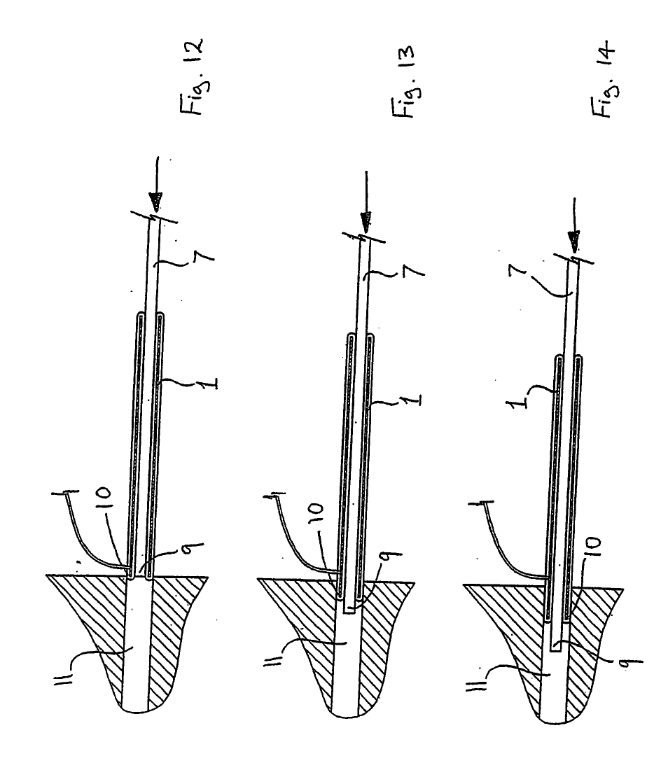

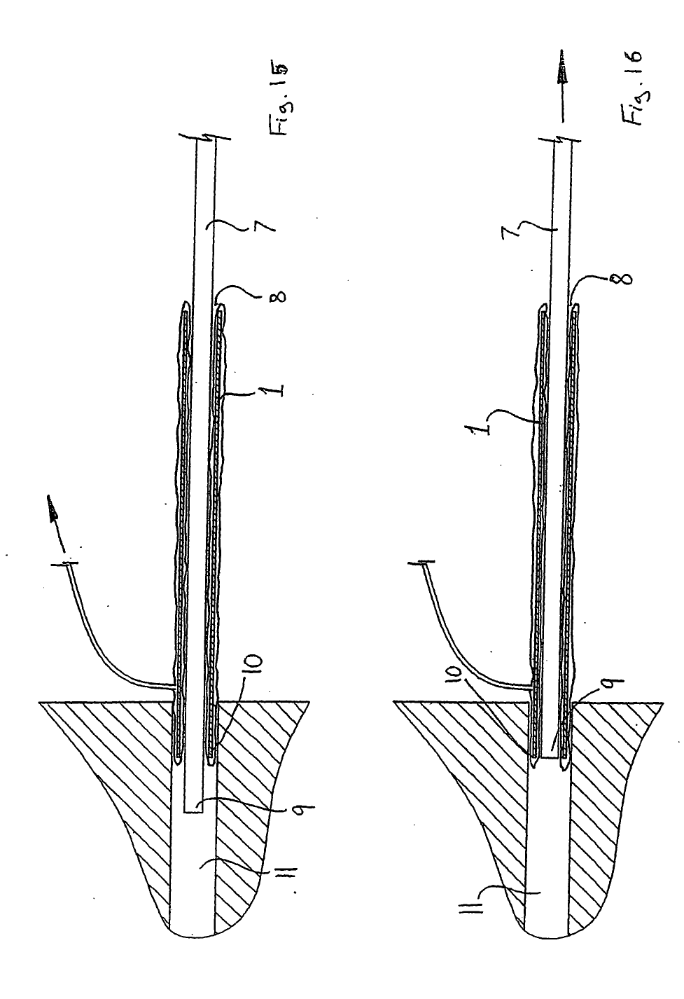

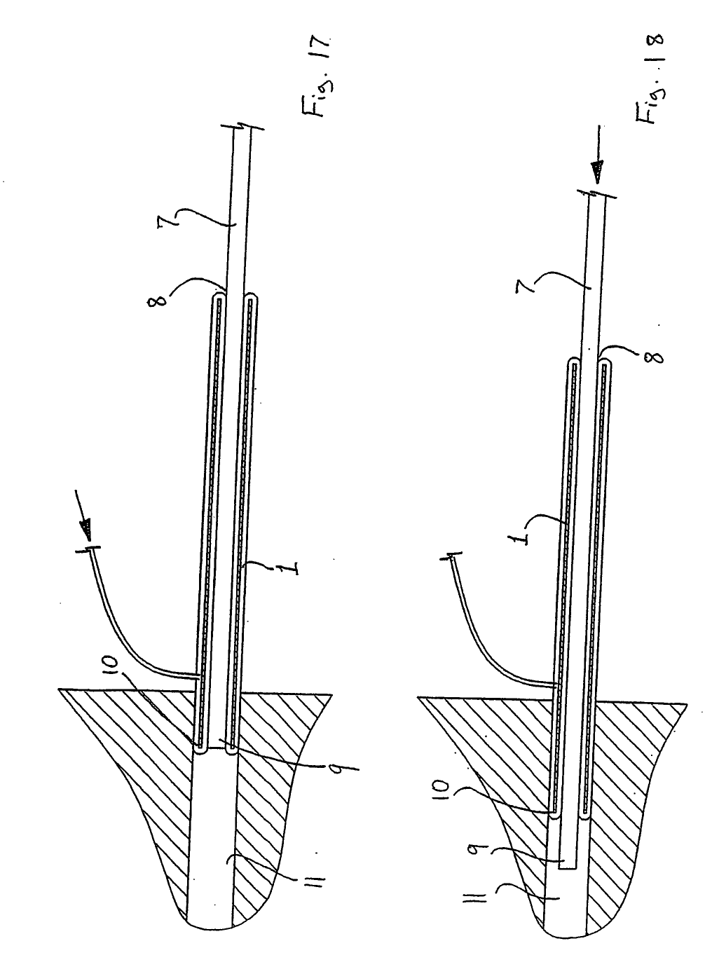

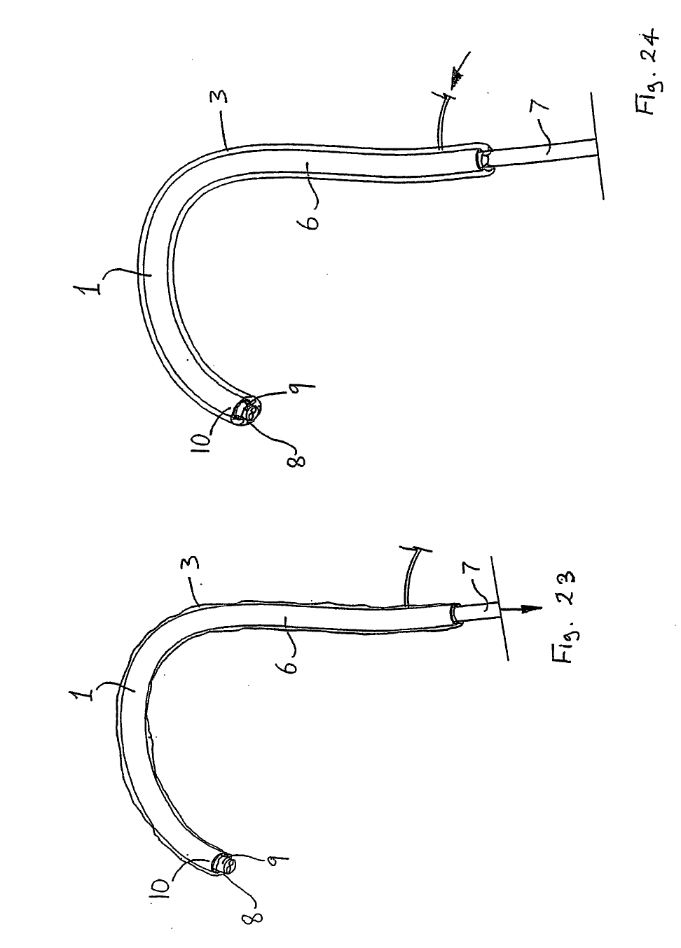

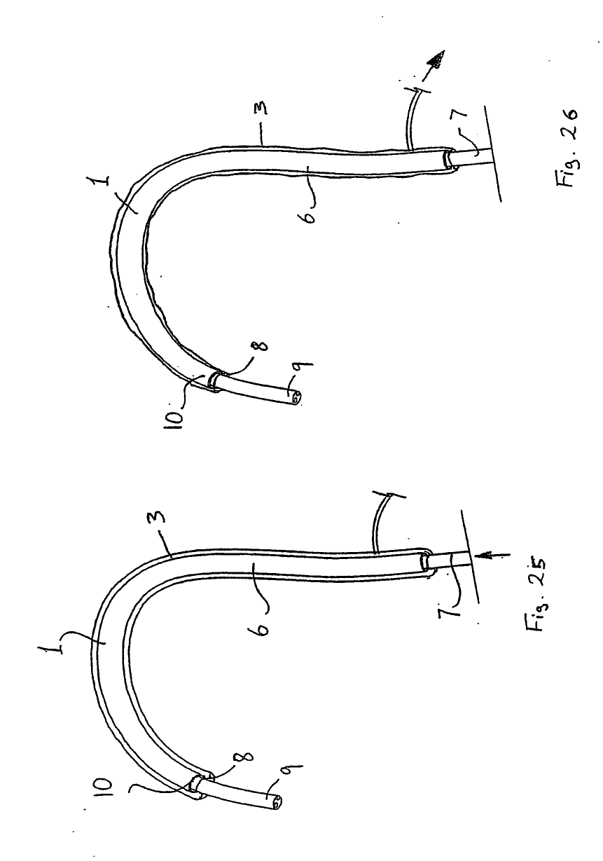

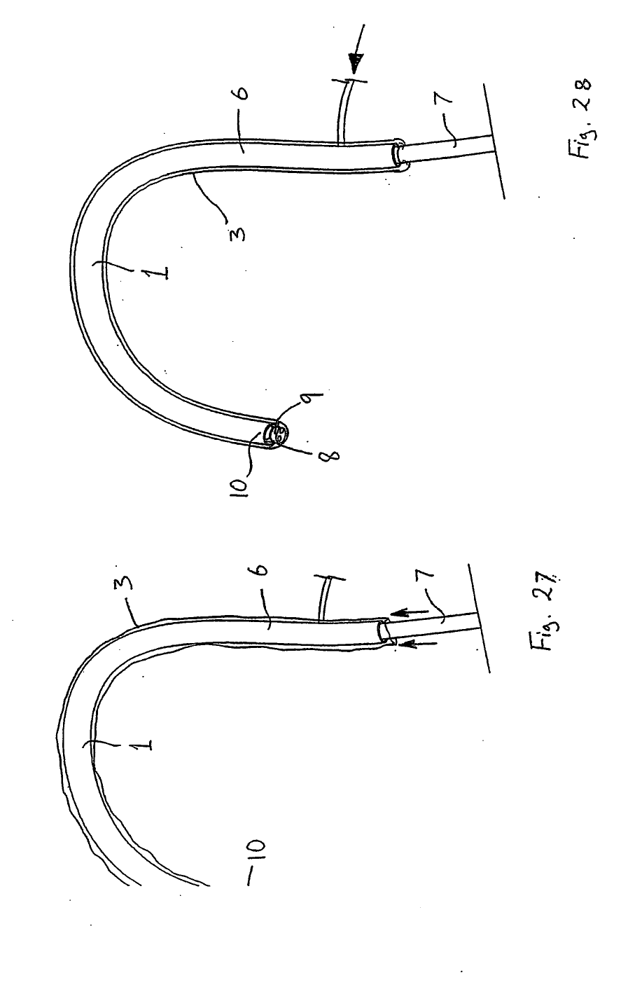

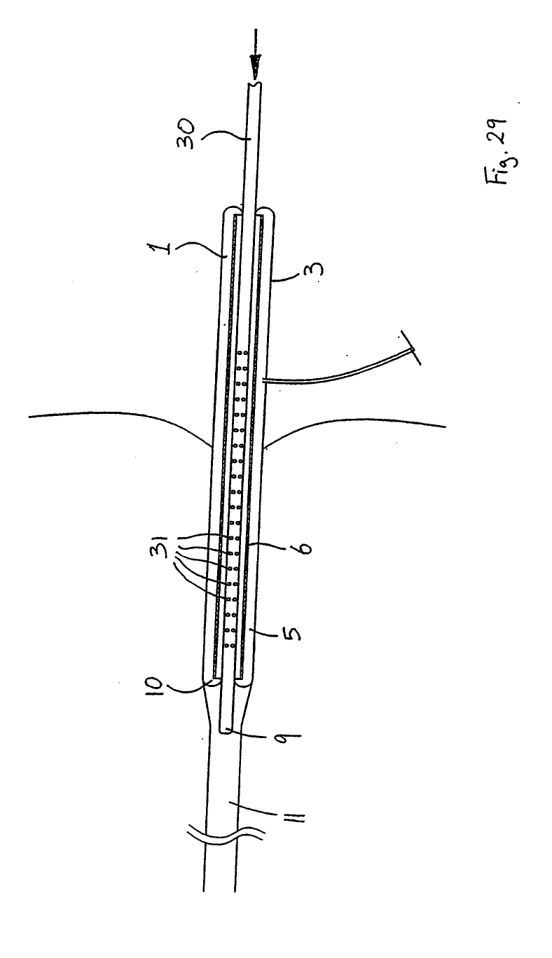

FIGS. 1 to 28 illustrate a particular embodiment of the device 1.

As illustrated in FIGS. 3 and 4, an outer wall 2 of the evertable tube 3 is connected at each end to an inner wall 4 of the tube 3, such that an enclosed inflation space 5 is defined between the walls 3, 4.

In this case, the device 1 has a tubular stiffening column 6, as illustrated in FIG. 9, to longitudinally stiffen the flexible tube 3. In this way, the stiffened device 1 may be advanced over the probe 7 in the lumen 8 when the tube 3 is deflated. The stiffening column 6 is located within the inflation space 5, and extends co-axially around the lumen 8.

In use, the probe 7 is inserted through the lumen 8 of the deflated tube 3 until the leading or distal end 9 of the probe 7 is aligned with the leading or distal end 10 of the guide device 1. The tube 3 is then inflated to grip the probe 7, and the probe 7 and guide device 1 are now ready for insertion into the passageway 11 (FIG. 12).

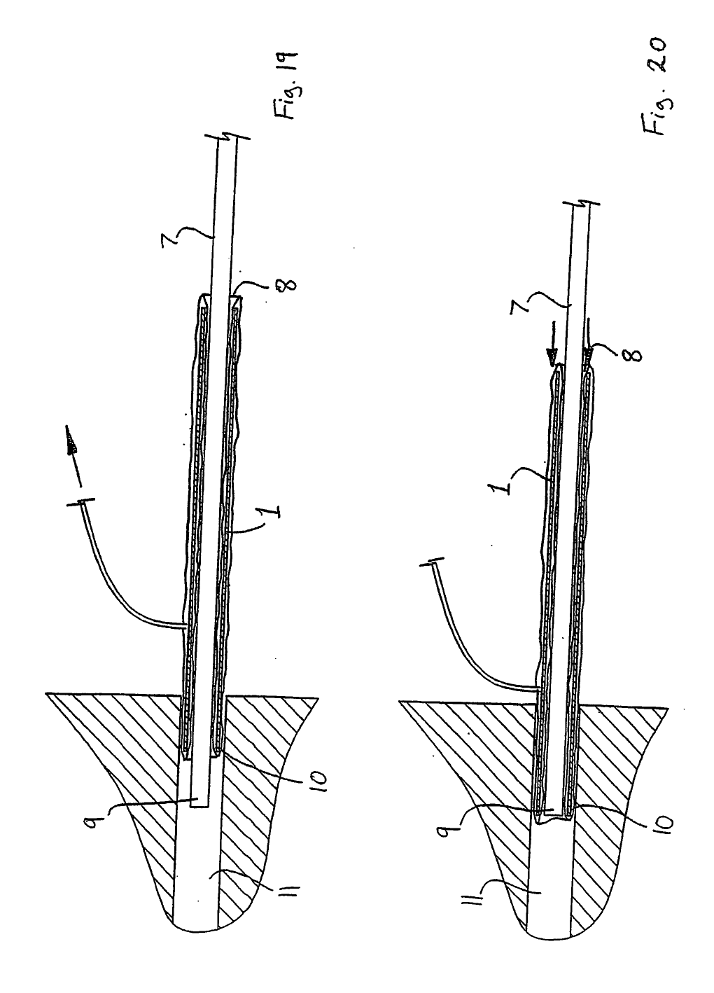

The probe 7 is advanced through the passageway 11 by pushing the probe 7 distally. Because the inflated tube 3 grips the probe 7, the tube 3 everts as the probe 7 advances distally through the passageway 11 (FIGS. 13 and 14).

The inflated tube 3 acts as a spacing means to prevent the probe 7 from engaging against the interior walls of the passageway 11 as the probe 7 advances through the passageway 11. In this manner, the probe 7 advances through the passageway 11 with a frictionless rolling action of the guide device 1 and with substantially no frictional contact between the probe 7 and the passageway 11.

As illustrated in FIGS. 12 to 14, due to the everting action of the guide device 1 with the probe 7, the leading end 9 of the probe 7 travels twice the distance of the leading end 10 of the guide device 1. Thus, the probe leading end 9 projects distally from the guide device 1.

To realign the two leading ends 9, 10, the tube 3 is deflated so that the probe 7 is no longer gripped by the tube 3 (FIG. 15), but there is still a central stiffening column 6 within the deflated tube 3. This allows the probe 7 to be retracted through the lumen 8 of the tube 3 until the leading ends 9, 10 are aligned (FIG. 16).

The tube 3 is re-inflated (FIG. 17), and the probe 7 is further advanced through the passageway 11 by pushing the probe 7 distally (FIG. 18).

The steps described above with reference to FIGS. 15 to 18 may be repeated as desired by the user to advance the probe 7 through the passageway 11 in an incremental manner.

In this way, the probe 7 advances through the passageway 11 in a “2 steps forward—1 step back” manner.

It will be appreciated that the guide device 1 may alternatively be advanced over the probe 7 when the tube 3 is deflated to align the leading end 10 of the guide device 1 with the leading end 9 of the probe 7, as illustrated in FIGS. 19 and 20. In this case, the probe 7 is maintained in a fixed position during realignment of the leading ends 9, 10.

Realignment of the leading ends 9, 10 by advancing the guide device 1 over the probe 7 has the advantage that all movement of the guide device 1 and the probe 7 is in the distal direction through the passageway 11. This provides for a smooth advancement procedure, which can be of particular importance when the probe is being advanced through a floppy or flexible passageway, such as a colon.

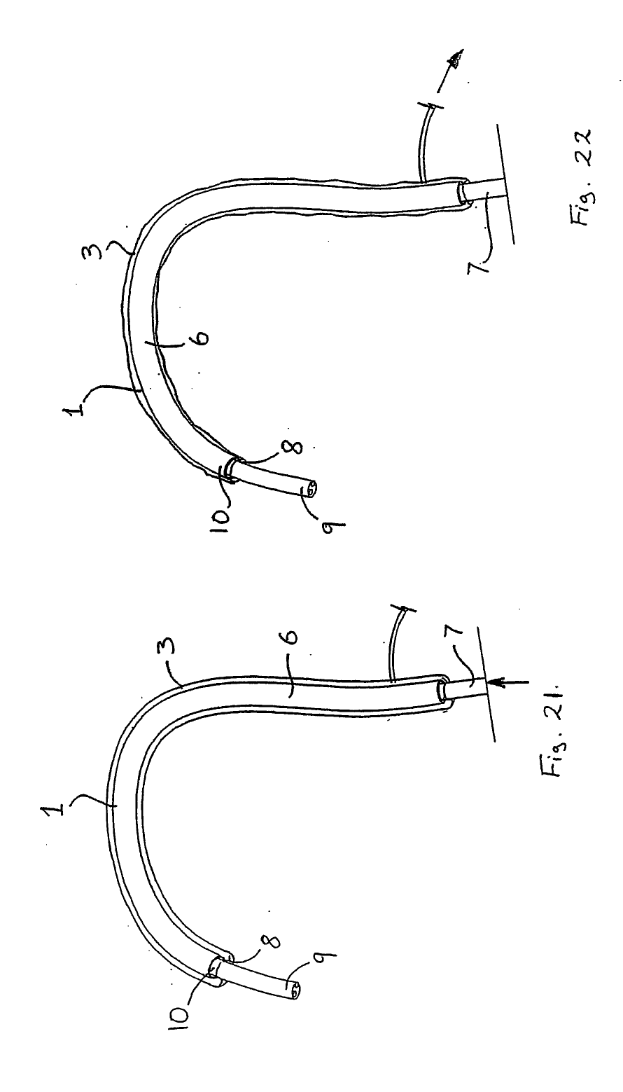

The guide device 1 is suitable for assisting the advancement of the probe 7 through curved or tortuous passageways such as illustrated in FIGS. 21 to 28.

FIGS. 21 to 28 illustrate advancement of the probe 7 through a curved passageway. The leading end 9 of the probe 7 can be aligned with the leading end 10 of the guide device 1 by retracting the probe 7 through the lumen 8 of the deflated tube 3 (FIGS. 21 to 24), or by advancing the deflated guide device 1 over the probe 7 (FIGS. 25 to 28), in a manner similar to that described previously with reference to FIGS. 12 to 20.

The stiffening column 6 may be a simple tube as illustrated in FIG. 9. Alternatively the stiffening column 21 may comprise at least one corrugation 20 on the column 21 to resist kinking of the column 21 as the guide device 1 bends around a curve in the passageway. The corrugation 20 may extend along the column 21 in a convoluted manner, as illustrated in the column 21 of FIG. 10, or the corrugation may extend around the column 21 in a loop.

A further alternative is illustrated in FIG. 11, in which a stiffening column 22 has a slit 23 through the column wall, the slit extending along the column 22 in a spiral. The slit column 22 is normally flexible such that when the tube 3 is in the inflated configuration, the column 22 provides minimum resistance to eversion of the tube 3 and minimum resistance to bending of the guide device 1 through the passageway. However, when the tube 3 is deflated it has been found that the column 22 becomes much stiffer, and thus it is relatively easy to manipulate the guide device 1 and the probe 7 to align the leading ends 9, 10, as described above.

It will be appreciated that a clamp may be provided to engage with an outer wall or an inner wall of the tube to selectively prevent tube eversion, and/or to selectively prevent advancement of the guide device over a probe in the lumen.

Referring to FIGS. 29 to 33 there is illustrated another probe 30 according to the invention in use with the guide device 1 described previously with reference to FIGS. 1 to 28.

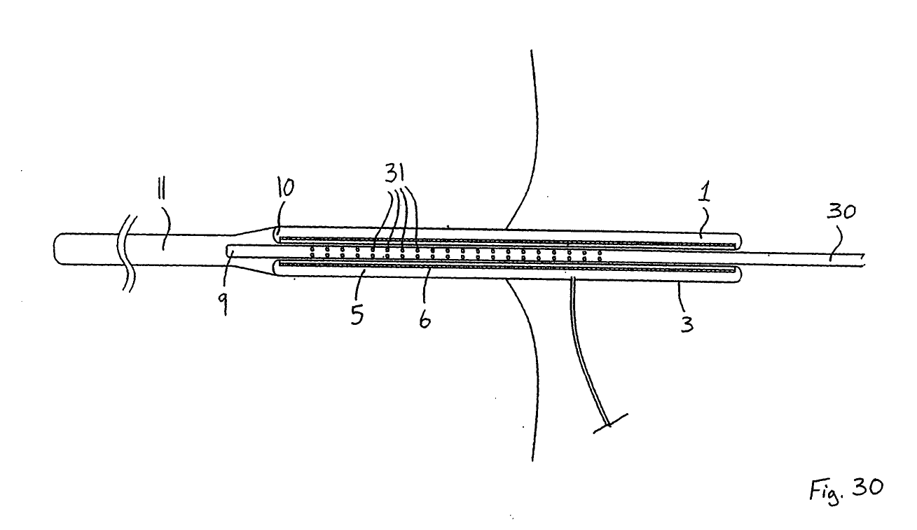

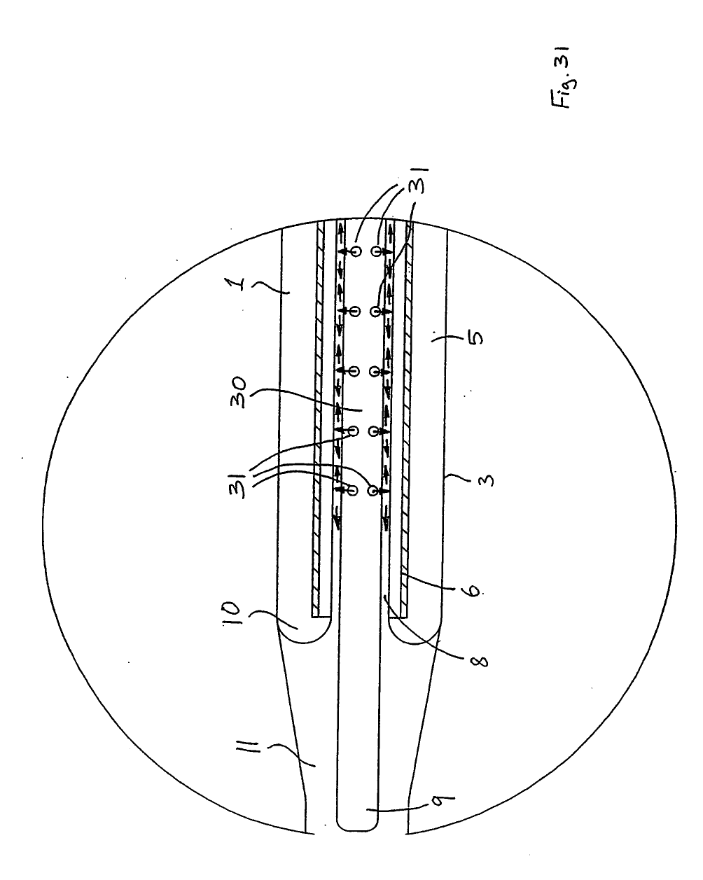

The probe 30 comprises a plurality of fluid openings 31 on an outer surface of the probe 30 for ejecting a fluid, such as air, through the openings 31. A fluid inlet lumen is provided extending through the probe 30 in communication with the openings 31 to facilitate passage of air from externally of the passageway 11, through the fluid inlet lumen and out through the openings 31, as illustrated in FIGS. 30 and 31. In this manner a fluid cushion may be created between the probe 30 and the guide device 1 to move the device 1 out of contact with the probe 30 without deflating the tube 3.

In use, the probe 7 is advanced through the passageway 11 with the associated eversion of the tube 3 (FIG. 29), in a manner similar to that described previously with reference to FIGS. 12 to 14.

To realign the two leading ends 9, 10, air is passed from externally of the passageway 11, through the fluid inlet lumen of the probe 30, and out through the openings 31 (FIGS. 30 and 31). The air exiting the openings 31 pushes the guide device 1 out of contact with the probe 30 to create a cushion of air in the central lumen 8 between the probe 30 and the device 1.

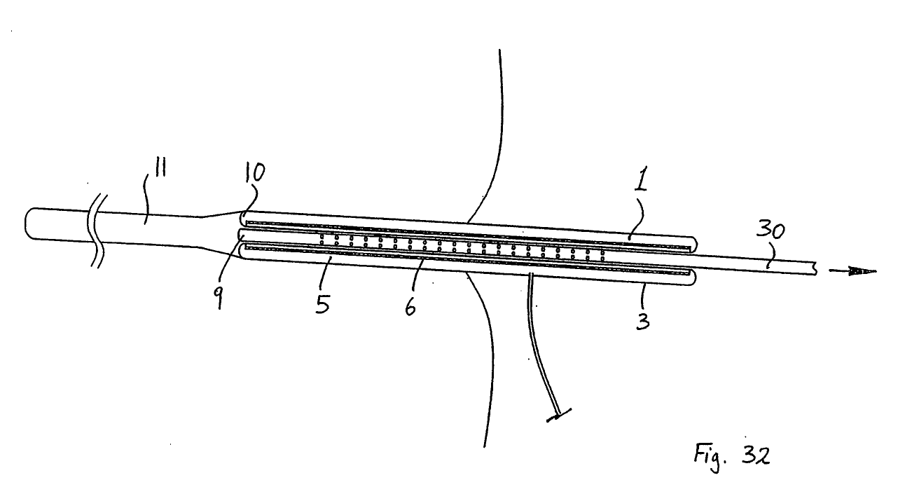

Because the guide device 1 is now out of contact with the probe 30, this enables the probe 30 to be retracted through the lumen 8 of the tube 3 until the leading ends 9, 10 are aligned (FIG. 32).

The passage of air out through the openings 31 is then ceased. This causes the cushion of air between the probe 30 and the device 1 to disperse, and the tube 3 grips the probe 30 again.

The steps described above with reference to FIGS. 29 to 32 may be repeated as desired by the user to advance the probe 30 through the passageway 11 in an incremental manner. In this way the probe 30 advances through the passageway 11 in a “2 steps forward—1 step back” manner.

It will be appreciated that the guide device 1 may alternatively be advanced over the probe 30 while the air cushion is between the probe 30 and the device 1 to align the leading end 10 of the device 1 with the leading end 9 of the probe 30. In this case the probe 30 is maintained in a fixed position during realignment of the leading ends 9, 10.

It is not necessary for the tube 3 to be deflated to align the leading end 9 of the probe 30 with the leading end 10 of the guide device 1.

Thus the inflated tube 3 maintains a grip on the wall of the passageway 11. This may be particularly advantageous in the case where the inflated tube 3 is pulled back while gripping the wall of a colon to at least partially straighten a previously looped section of colon, such as the sigmoid colon.

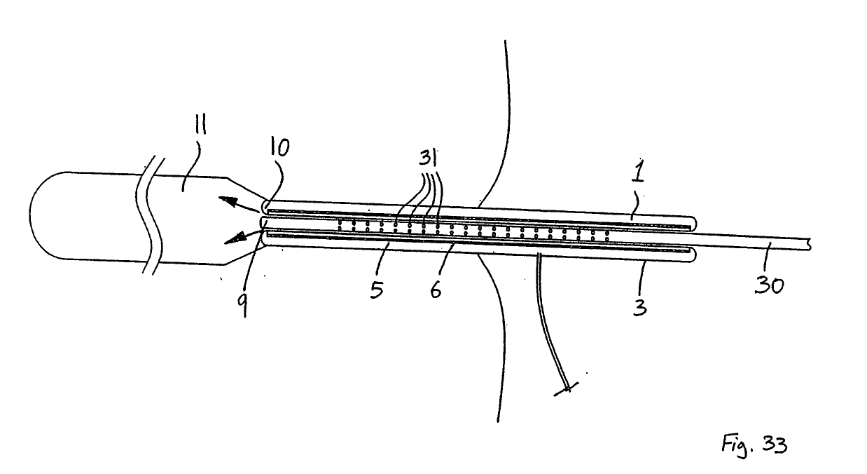

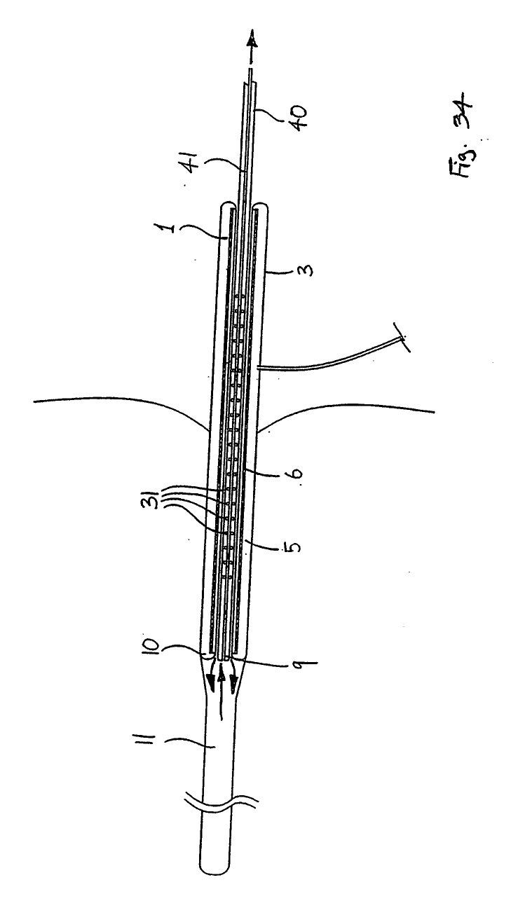

Depending on a number of factors, such as the volume of the passageway 11, the volumetric flow rate of the air, the duration of time for which air is passed out through the fluid openings 31, the passageway 11 may in certain circumstances become distended or bloated as a result of the air being passed out through the fluid openings 31, as illustrated in FIG. 33. In the case when the passageway 11 is a colon, this is a potentially painful and/or dangerous occurrence.

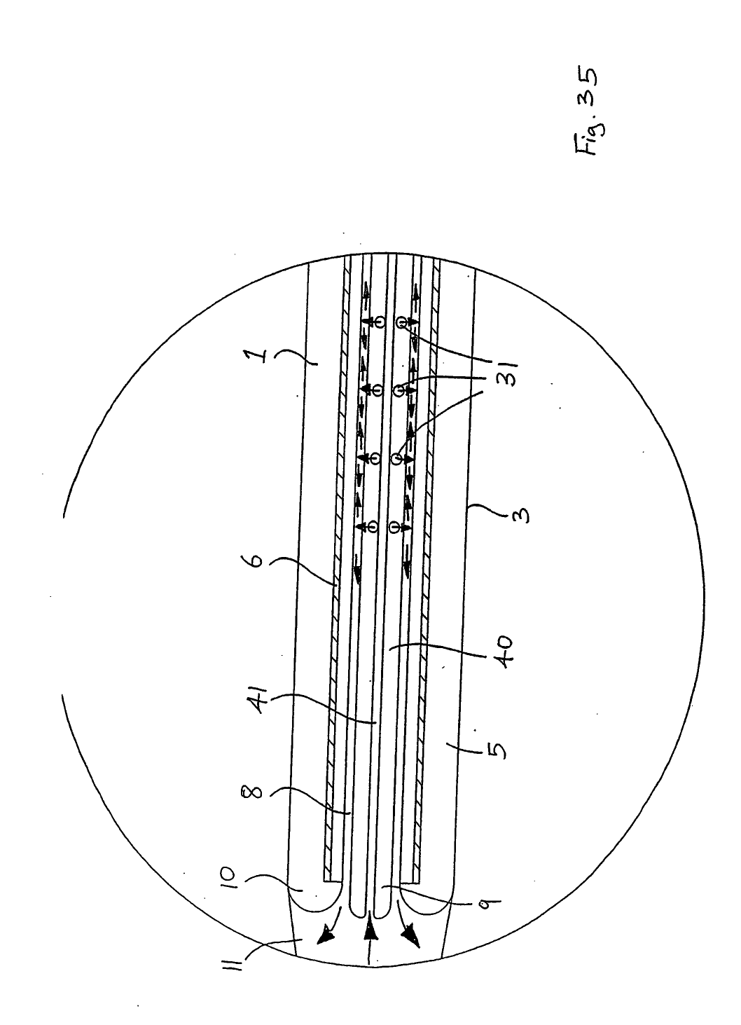

In FIGS. 34 and 35 there is illustrated another probe 40 according to the invention, which is similar to the probe 30 of FIGS. 29 to 33, and similar elements in FIGS. 34 and 35 are assigned the same reference numerals.

In this case, the probe 40 comprises a fluid exhaust lumen 41 extending therethrough. In this manner the fluid exhaust lumen 41 provides a means for any excess air in the passageway 11 to escape from the passageway 11, thereby preventing the passageway 11 from becoming distended or bloated. In particular it is not necessary for the tube 3 to be deflated, or for the cushion of air between the probe 40 and the guide device 1 to be dispersed to enable the excess air in the passageway 11 to escape.

Suction may be applied to the fluid exhaust lumen 41 to further assist in the removal of excess air from within the passageway 11.

It will be understood that fluids other than air may alternatively be used to create the fluid cushion between the probe and the guide device. For example water could be used to create the fluid cushion.

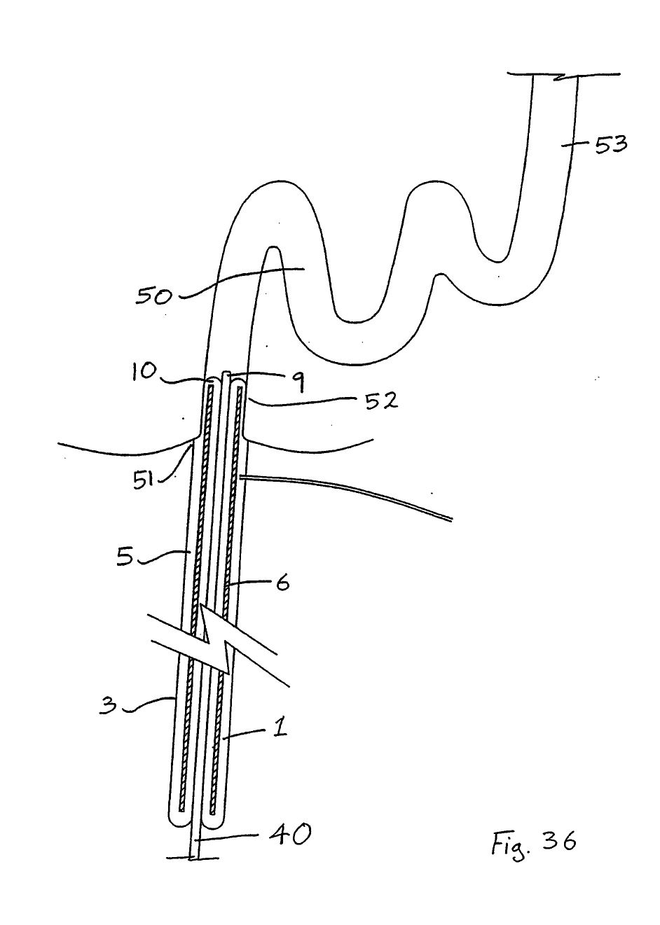

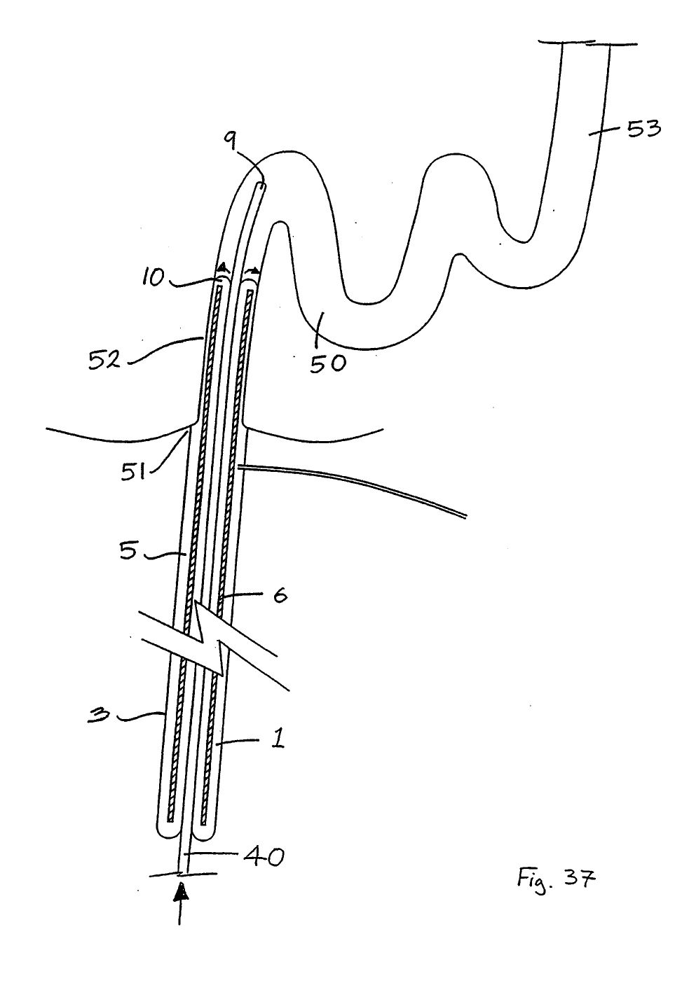

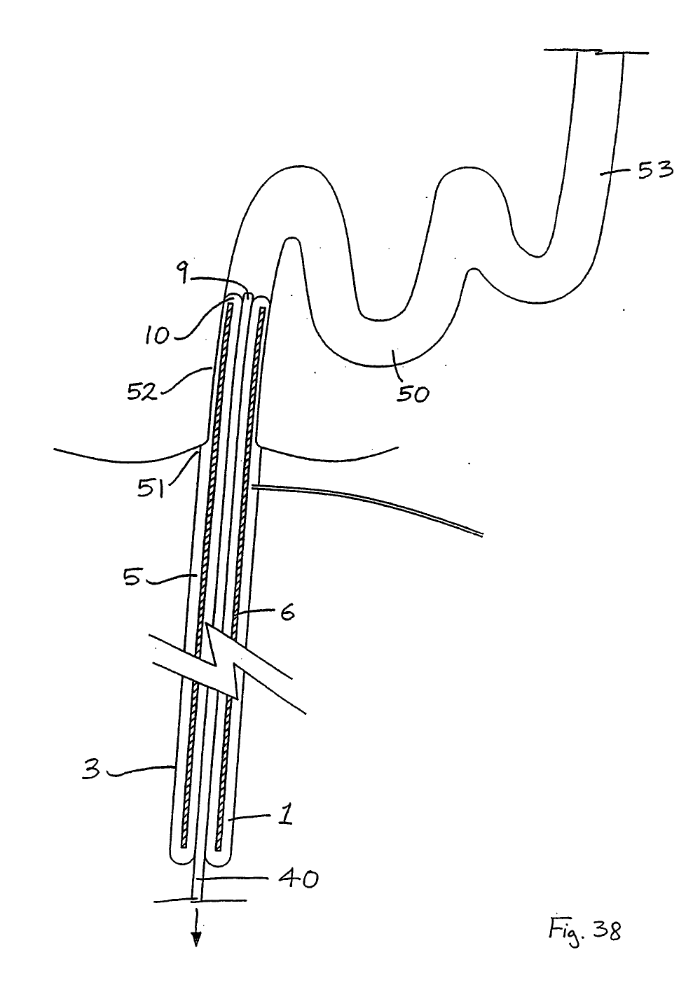

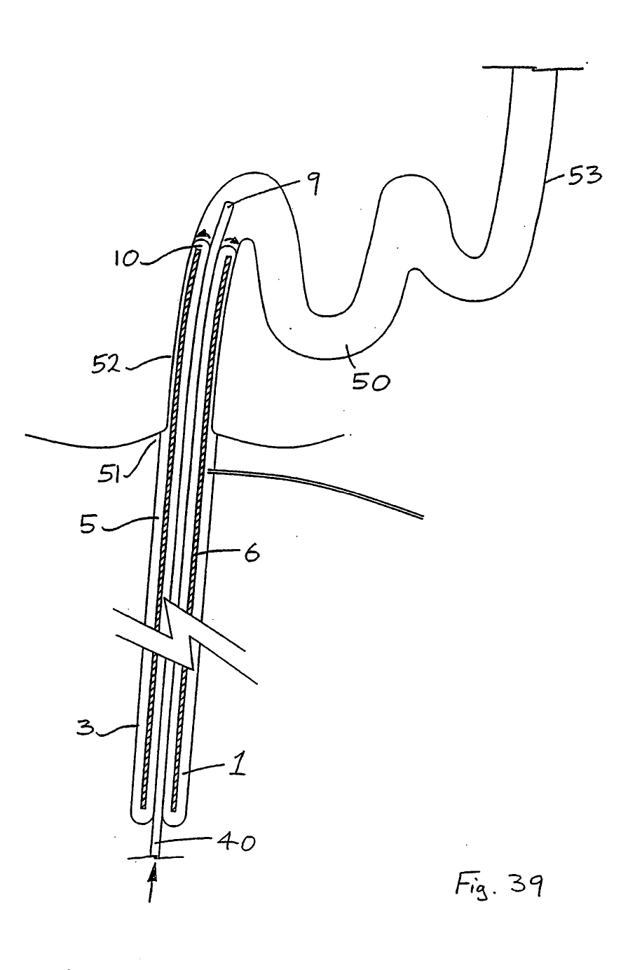

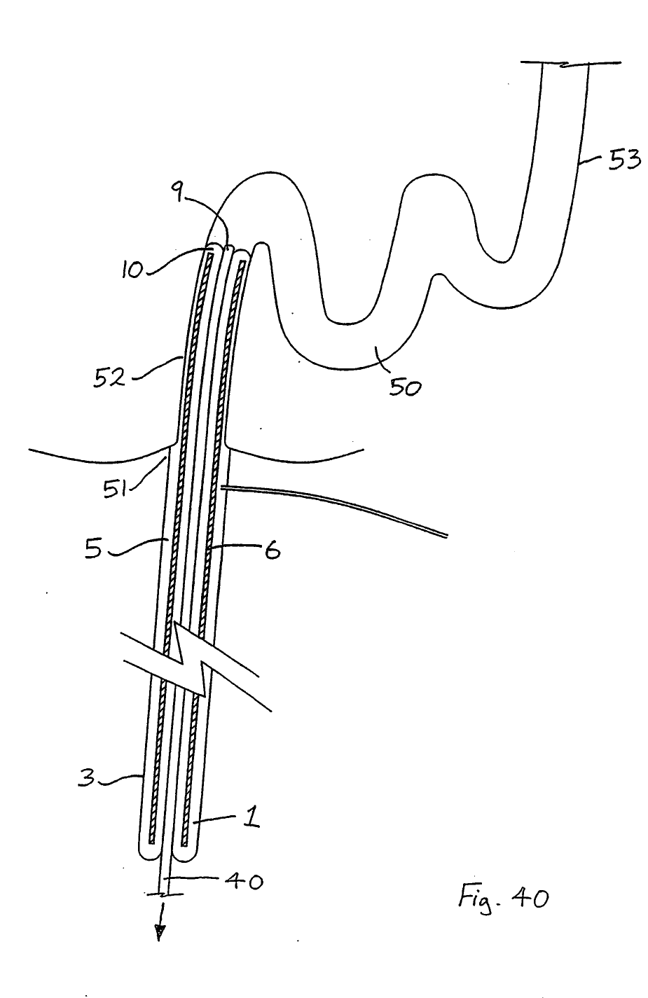

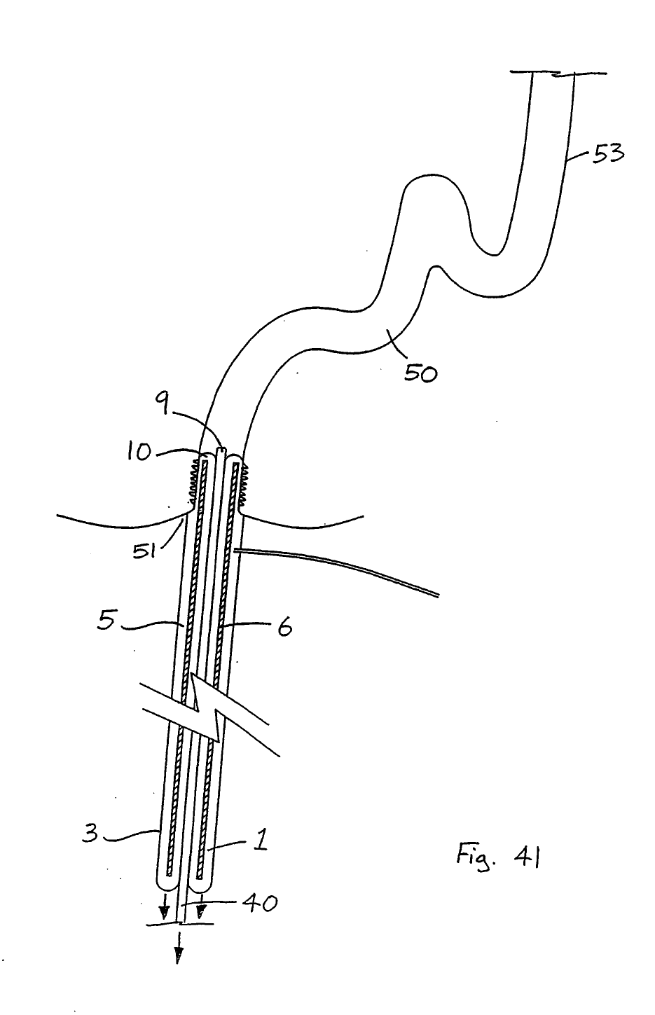

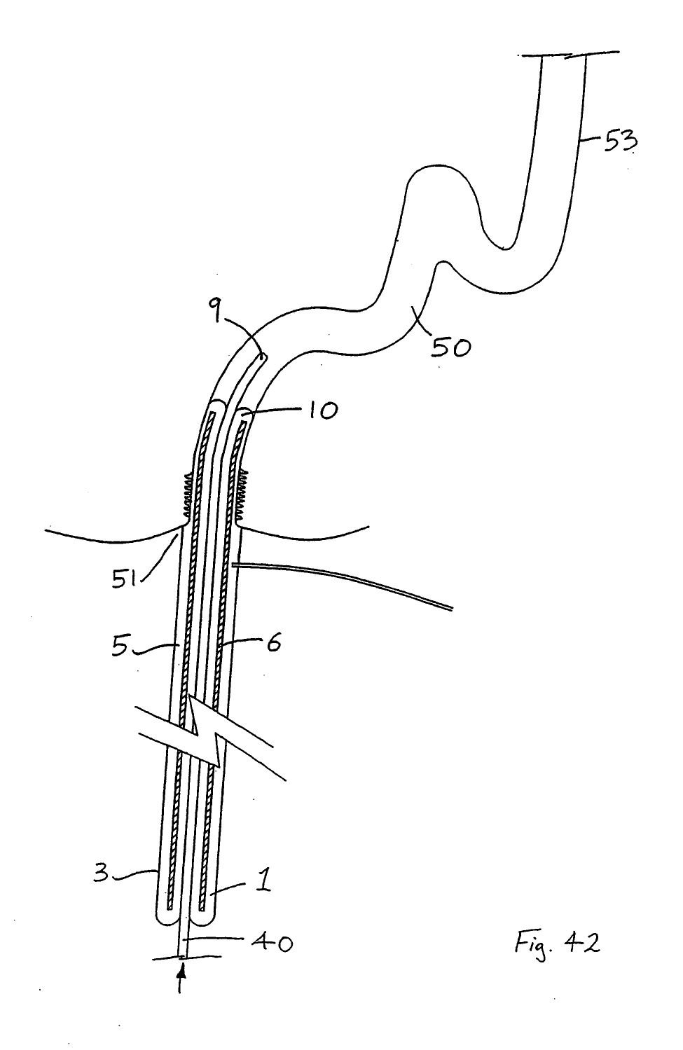

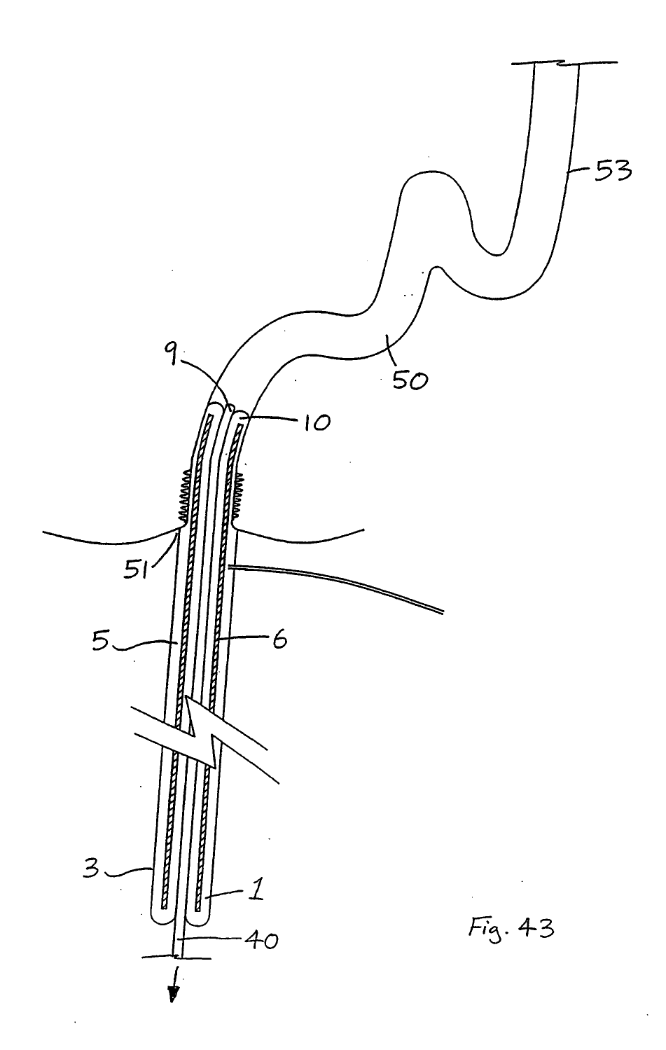

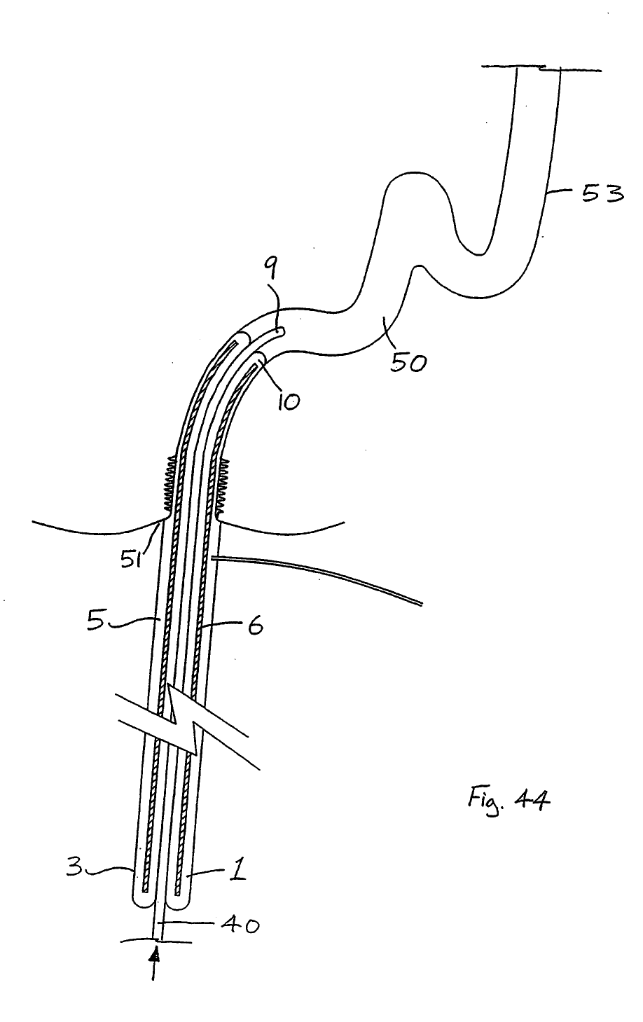

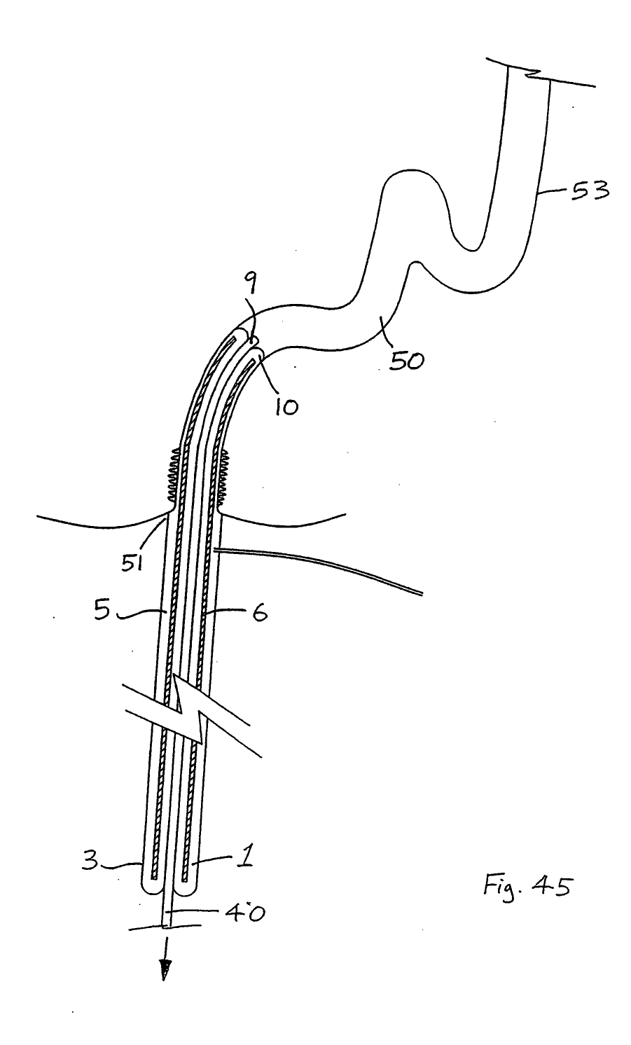

FIGS. 36 to 48 illustrate the guide device 1 and the probe 40 being used to straighten a looped sigmoid colon 50. The probe 40 may in one case be a colonoscope for advancement through a colon.

The probe 40 is first inserted through the lumen 8 of the deflated tube 3 until the leading end 9 of the probe 40 is aligned with the leading end 10 of the guide device 1. The tube 3 is then inflated to grip the probe 40, and the probe 40 and the guide device 1 are now ready for insertion into the anus 51 of the patient (FIG. 36).

The probe 40 is advanced through the rectum 52 by pushing the probe 40. Because the inflated tube 3 grips the probe 40, the tube 3 everts as the probe 40 advances distally through the rectum 52 (FIG. 37).

The inflated tube 3 also grips the interior wall of the colon, thereby anchoring the interior wall of the colon to the guide device 1.

To realign the leading ends 9, 10, air is passed from externally of the colon, through the fluid inlet lumen of the probe 40, and out through the openings 31 to create the cushion of air between the probe 40 and the guide device 1.

The probe 40 is then retracted through the lumen 8 of the tube 3 while maintaining the position of the guide device 1 fixed until the leading ends 9, 10 are aligned (FIG. 38). The passage of air out through the openings 31 is ceased and the tube 3 re-grips the probe 40.

The steps of advancement of the probe 40 and realignment of the leading ends 9, 10 may be repeated in an incremental manner (FIGS. 39 and 40) to advance the probe 40 through the rectum 52 to the proximal end of the sigmoid colon 50.

Next, the probe 40 and the guide device 1 are both retracted. Because the interior wall of the colon is anchored to the guide device 1, this action causes part of the wall of the colon to be accordioned down, and thereby causes the sigmoid colon 50 to be partially straightened (FIG. 41). During this straightening step there is no cushion of air between the probe 40 and the guide device 1, and the tube 3 grips the probe 40.

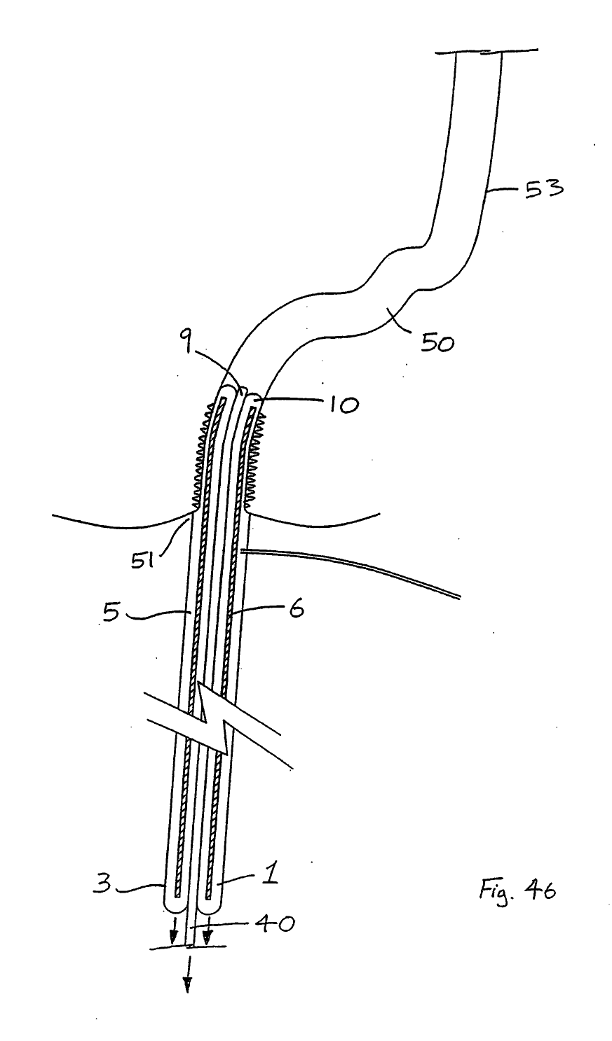

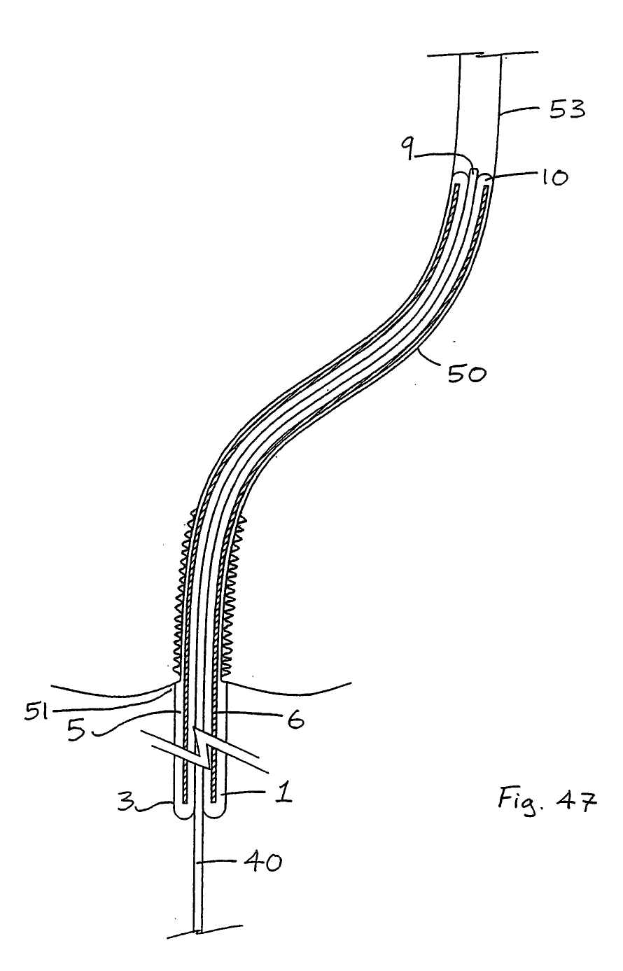

The steps of advancement of the probe 40 and realignment of the leading ends 9, 10 may be repeated in an incremental manner (FIGS. 42 to 45) to advance the probe 40 further through the partially straightened sigmoid colon 50. The probe 40 and the guide device 1 are then retracted to accordion down a further part of the wall of the colon, and thereby further straighten the sigmoid colon 50 (FIG. 46).

This process of advancement of the probe 40, realignment of the leading ends 9, 10, and straightening of the sigmoid colon 50 may be repeated in an incremental manner until the leading end 10 of the guide device 1 has reached the proximal end of the descending colon 53 and the sigmoid colon 50 has been fully straightened (FIG. 47).

The guide device 1 and the probe 40 may be used to collapse the sigmoid colon of a patient to a reduced, straightened configuration substantially without causing stretching of the colon and the mesentery to which the colon is attached, and causing the resultant pain and discomfort to the patient.

It is not necessary to advance the probe 40 all the way through the sigmoid colon to the proximal end of the descending colon before beginning the reduction of the sigmoid colon using the guide device 1 and the probe 40.

Because the tube 3 is not deflated upon realignment of the leading ends 9, 10, this ensures that the grip exerted by the inflated tube 3 on the interior wall of the colon is maintained throughout the straightening procedure. In particular, the accordioned part of the colon wall will remain accordioned down during realignment of the leading ends 9, 10.

The process of advancement of the probe 40, realignment of the leading ends 9, 10, and straightening of the sigmoid colon 50 involves the steps of advancement of the probe 40, retraction of the probe 40, retraction of the guide device 1 and passing air through the fluid inlet lumen of the probe 40 repeated in a desired sequence. This process could therefore be automated in certain cases to achieve straightening of the colon in a relatively fast, painless manner.

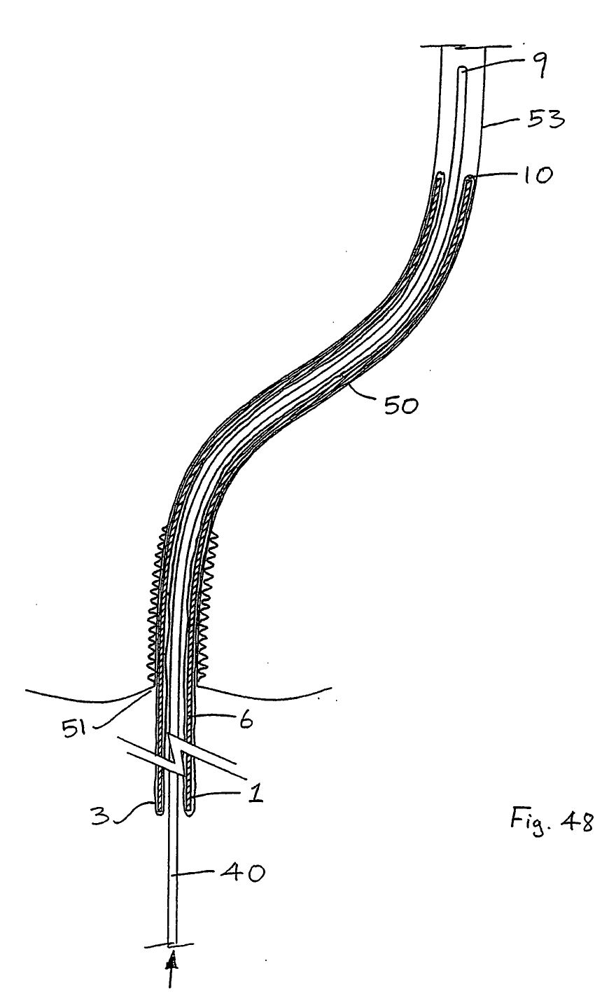

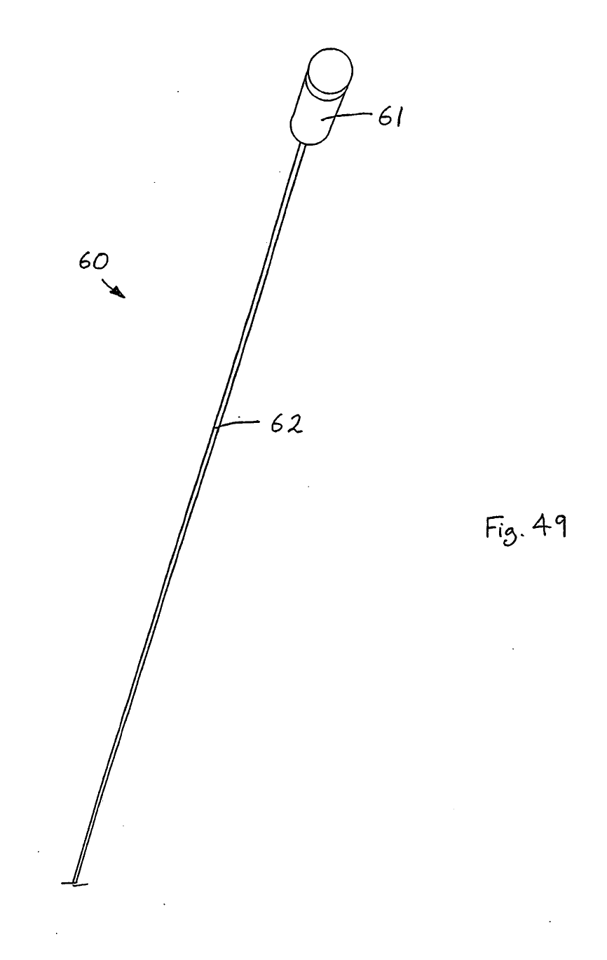

When the leading end 10 of the guide device 1 has reached the proximal end of the descending colon 53, the tube 3 is deflated. The probe 40 may then be advanced further distally through the descending colon 53 (FIG. 48) and into the transverse colon. The stiffening column 6 of the guide device 1 acts as a splint to maintain the sigmoid colon 50 in the straightened configuration. The splinting column 6 ensures that further advancement of the probe 40 through the descending colon 53 and into the transverse colon is possible by preventing loops from reforming in the sigmoid colon 50. In this manner, the column 6 minimises the pain or discomfort experienced by the patient during this procedure.

In this regard the stiffening column 6 is similar to the colonic overtube described in International patent application number PCT/IE02/00029, the relevant contents of which are incorporated herein by reference.

Means may be provided for opening up or ripping the tube 3 to enable the user to remove the tube 3 from the colon while the stiffening column 6 remains in position splinting the straightened sigmoid colon 50.

It has been found that by straightening the sigmoid colon 50 using the guide device 1 as described previously with reference to FIGS. 36 to 48, manual steering of the probe 40 during the incremental advancement through the colon is not required. Thus, an alternative vision system 60, as illustrated in FIG. 49, could be used with the guide device 1 to straighten the sigmoid colon 50.

The vision system 60 comprises a head 61 containing the viewing/lighting means to facilitate visualisation of the colon, and a thin body 62.

Because steering capabilities are not required for the vision system 60, the body 62 has a particularly small diameter for ease of retraction through the lumen 8 of the tube 3 for realignment of the leading ends 9, 10. In addition the body 62 may be of a low friction material for ease of retraction through the lumen 8 of the tube 3.

The head 61 may be similar to a video pill.

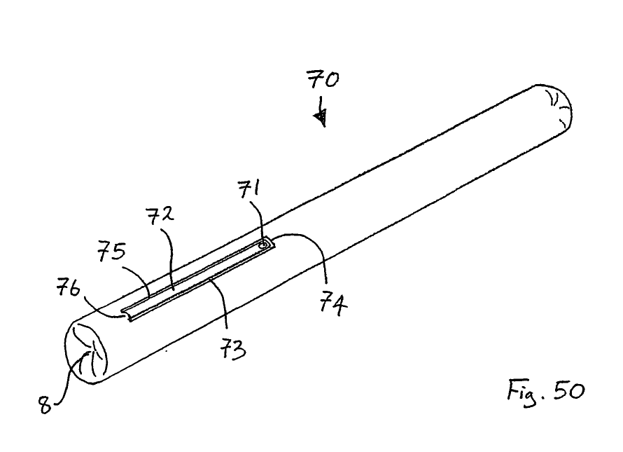

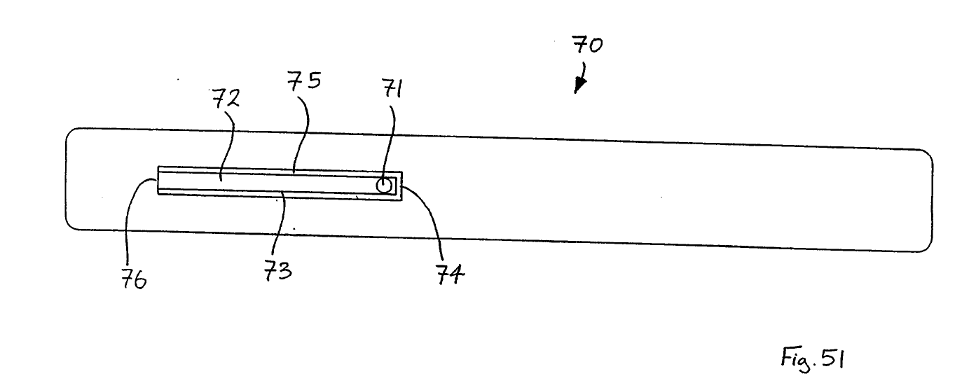

Referring to FIGS. 50 to 53 there is illustrated an evertable tube 70 according to the invention, which is similar to the tube 3 of the guide device 1 of FIGS. 1 to 28, and similar elements in FIGS. 50 to 53 are assigned the same reference numerals.

In this case no stiffening column is provided within the inflation space 5.

The tube 70 has an inflation port 71 for inflating the tube 70 to grip a member, such as a probe, in the lumen 8. Advancement of the member, for example through a colon, will then cause eversion of the tube 70. In this case the inflation port 71 is provided in the form of an aperture in the outer wall 2 of the tube 70.

A sheath 72 is fixedly attached to the outer wall 2 of the tube 70 by welding three sides 73, 74, 75 of the sheath 72 to the wall 2. The sheath 72 extends from the inflation port 71 along the wall 2 of the tube 70 parallel to the longitudinal axis of the tube 70 to an open end 76 of the sheath 72.

To inflate the tube 70, air is passed, for example using a hand pump 77, through the open end 76 of the sheath 72, along the sheath 72 between the wall 2 of the tube 70 and the sheath 72, through the inflation port 71 and into the inflation space 5 (FIG. 52).

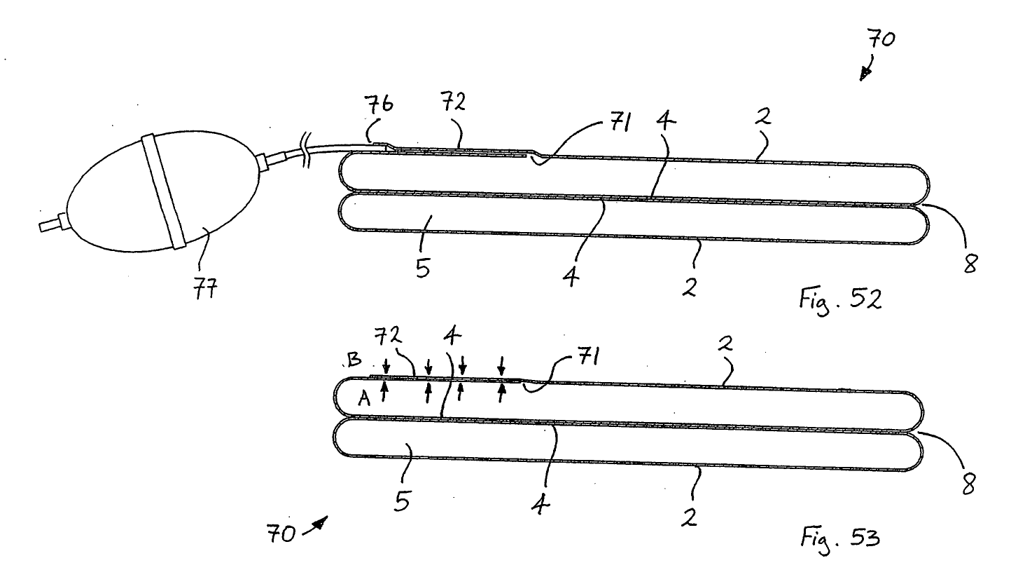

When the tube 70 has been inflated, the pump 77 is removed from the open end 76 of the sheath 72. The air pressure A within the inflation space 5 acting on the wall 2 of the tube 70, and the atmospheric pressure B acting on the sheath 72 combine to press the sheath 72 tightly against the wall 2 of the tube 70 (FIG. 53).

In this manner the sheath 72 acts as a self-closing valve to prevent leakage of air from within the inflation space 5 out through the inflation port 71.

In addition the low-profile sheath 4 presses tightly against the wall 2 of the tube 70 when the tube 70 is inflated, as illustrated in FIGS. 50 and 53. This ensures complete eversion of the tube 70 is possible.

It will be appreciated that the sheath 72 may be fixed to the wall 2 of the tube 70 by any suitable means, such as by adhesive bonding. The sheath 72 may alternatively be integrally formed with the wall 2.

The fixing means may be reinforced in the region of the open end 76 of the sheath 72.

The sheath 72 may be of the same or a different material to the wall 2 of the tube 70.

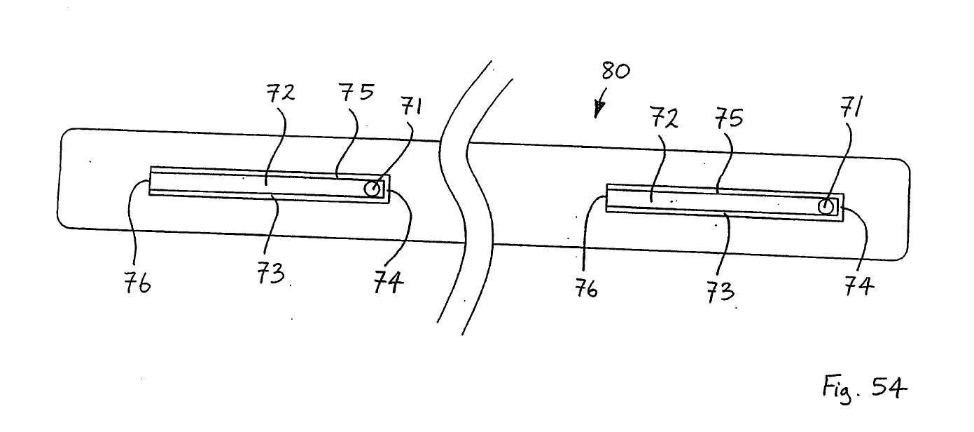

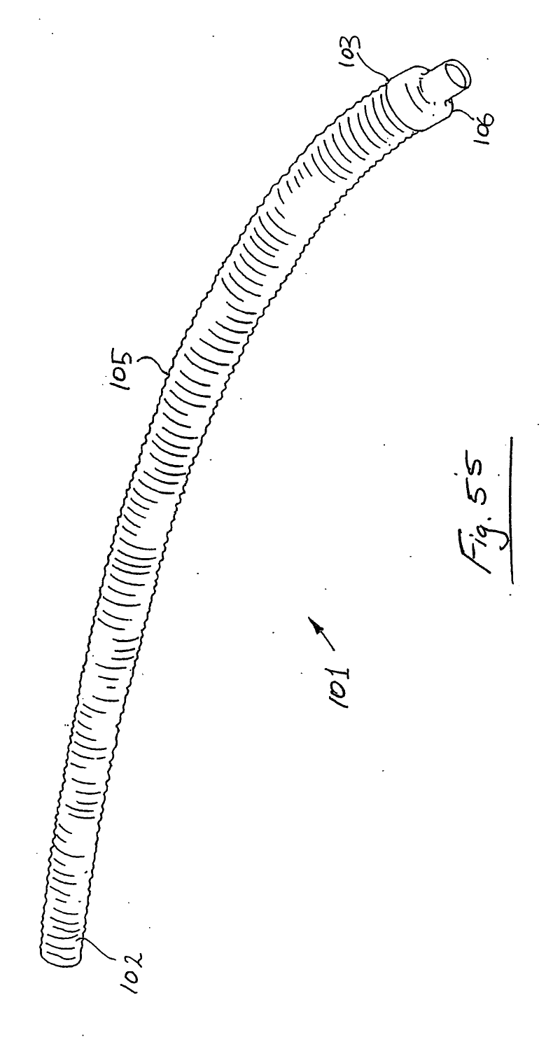

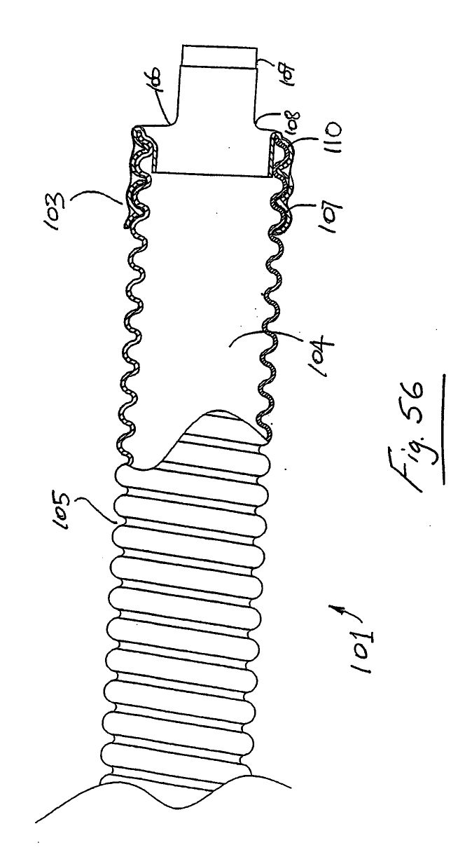

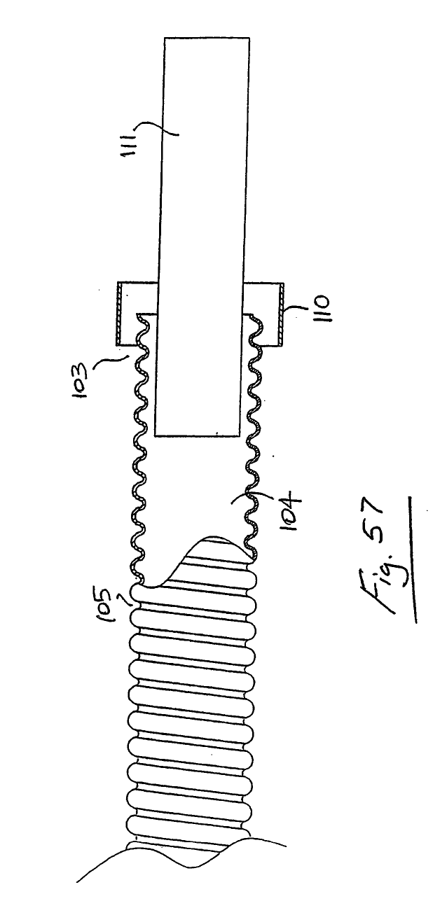

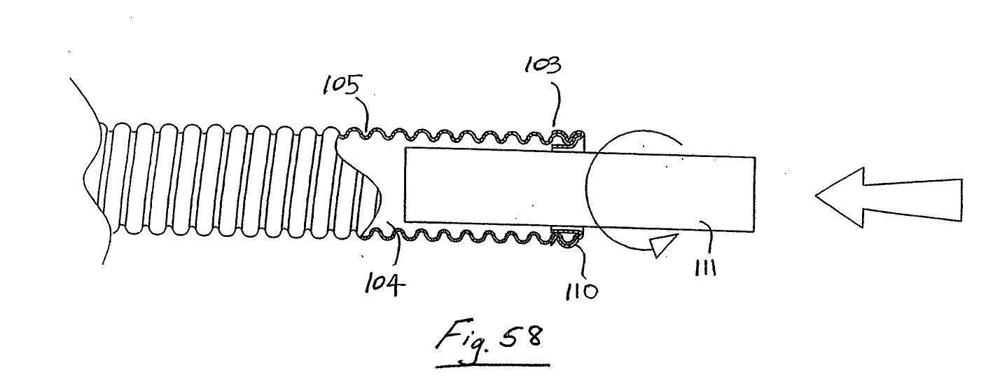

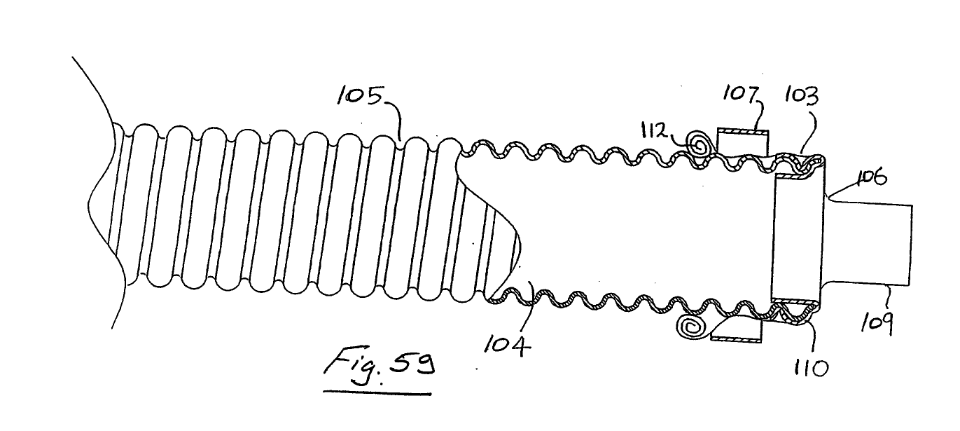

More than one inflation port 71 may be provided in the wall 2, as illustrated in the evertable tube 80 of FIG. 54. At each inflation port 71 a self-closing sheath valve 72 is preferably provided.

Because of the everting motion of the tube 80, the inflation ports 71 move from being on the outer surface of the tube 80 into the lumen 8 along the inner surface of the tube 80. By providing more than one inflation port 71, this increases the possibility of a port 71 being located along the outer surface of the tube 80 when it is desired to inflate the tube 80. In particular in certain circumstances it may be essential to inflate or deflate the tube 80 without everting or moving the tube 80, for example when the tube 80 is in situ in a colon. In such cases the multi-inflation port configuration of the tube 80 is particularly advantageous for providing easy and fast access to an inflation port 71.

It is to be understood that in another case a stiffening column may be located within the inflation space 5 of the tube 70. In this case the tube 70 and stiffening column may be used as a guide device in a manner similar to the guide device 1 described previously.

It will be appreciated that the inflation port configuration described with reference to FIGS. 50 to 53 may be applied with a variety of inflatable evertable devices, such as an exsanguinator, an invaginator, an introducer device, or a hand-access device to allow surgical procedure to be converted from an open procedure into a hand-assisted laparoscopic procedure.

The invention is not limited to the embodiments hereinbefore described, with reference to the accompanying drawings, which may be varied in construction and detail.

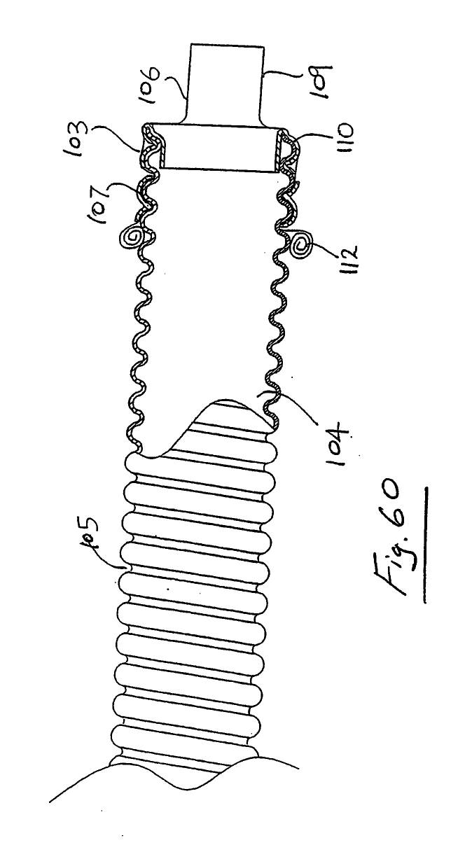

Referring to the drawings and initially to FIGS. 55 to 77 thereof, there is illustrated a colonic overtube 101 suitable for use in a method of performing a colonoscopy according to the invention. The overtube 101 may be used to maintain a section of a colon, such as a transverse colon or a sigmoid colon, in a straightened configuration.

The overtube 101 has a proximal end 102 for location, in use, externally of a colon, and a distal end 103 for insertion into a colon. A typical length for the overtube 101 is 0.5 m.

A colonoscope lumen 104 extends through the overtube 101 to facilitate passing the overtube 101 over a colonoscope. At least a portion of the overtube 101 is laterally flexible. In this manner the overtube 101 may flex substantially without kinking during advancement of the overtube 101 through a colon. In this case and as illustrated in FIGS. 55 and 56, the overtube 101 defines a corrugation 105 which is convoluted, the corrugation 105 extending along the entire length of the overtube 101 from the proximal end 102 to the distal end 103. The corrugated configuration of the overtube 101 minimises the possibility of the overtube 101 kinking as the overtube 101 is advanced over a colonoscope through a colon. As illustrated in particular in FIG. 56, in this case the corrugation 105 is provided on both the interior surface and the exterior surface of the overtube 101.

A flexible seal is provided at the distal end 103 of the overtube 101 for sealing between the overtube 101 and a colonoscope extending through the colonoscope lumen 104. The seal is in the form of a tubular sheath 106 of film material, in this case silicone, which is fixed to an exterior surface of the overtube 101 at the distal end 103 of the overtube 101 by means of a section of heat-shrink tubing 107. As illustrated in FIG. 56, the sheath 106 extends inwardly at the distal end 103 of the overtube 101 for sealing between the overtube 101 and a colonoscope, and then distally of the distal end 103 of the overtube 101.

The sealing sheath 106 can evert from this distally extending configuration to a proximally extending configuration upon movement of the colonoscope relative to the overtube 101. This ensures a relatively large area of contact between the sheath 106 and the colonoscope which results in a secure seal between the colonoscope and the overtube 101.

The sheath 106 is folded over to define an inner sealing layer 109, and an outer sealing layer 108 around the inner sealing layer 109. The heat-shrink tubing 107 is provided between the inner and outer layers 109, 108 (FIG. 56).

The flexible nature of the seal 106 enables the seal 106 to adapt itself to the size of the colonoscope extending through the colonoscope lumen 104. In this manner, a secure, effective seal between the overtube 101 and a colonoscope is achieved regardless of the size diameter range of the colonoscope. In addition, the film seal 106 has a very low profile which facilitates easier passage of the overtube 101 over a colonoscope through a colon, while minimising the resultant discomfort to the patient.

The overtube 101 comprises another section of heat-shrink tubing 110 fixed to an exterior surface of the overtube 101 at the distal end 103 of the overtube 101. The tubing 110 extends around the distal end 103 of the overtube 101 partially into the colonoscope lumen 104 to define a rounded tip at the distal end 103 of the overtube 101. In this manner, the rounded tip tubing 110 ensures that there are no sharp edges at the distal end 103 of the overtube 101 for atraumatic advancement of the overtube 101 through a colon. The distal end 103 of the overtube 101 may be rounded off in a variety of different ways, such as by a separately mountable tip, or during the manufacturing process.

The overtube 101 is of a material which is thermally stable in use in a colon. In this case the thermally stable material used for the overtube 101 is polytetrafluoroethylene (PTFE)

In this manner, the overtube 101 is not overly stiff so that insertion of the overtube 101 into a colon, and navigation of the overtube 101 through a colon may be achieved without undue difficulty, and without causing undue discomfort to a patient. However once inserted into the colon, the stiffness of the overtube 101 remains above the minimum threshold of stiffness required to maintain a section of colon in a straightened configuration, and to prevent sigmoid loops from reforming as a colonoscope is passed through the colonoscope lumen 104.

A coating of a lubricious material such as a gel, for example a gel of silicone or polytetrafluoroethylene (PTFE) may be applied around the interior and/or exterior surfaces of the overtube 101 before use for ease of passage of the overtube 101 relative to a colonoscope and/or relative to a colon. Alternatively the coating of lubricious material may be provided as part of the overtube 101, such as by fixing the coating to the overtube 101, or by providing the coating integral with the overtube 101.

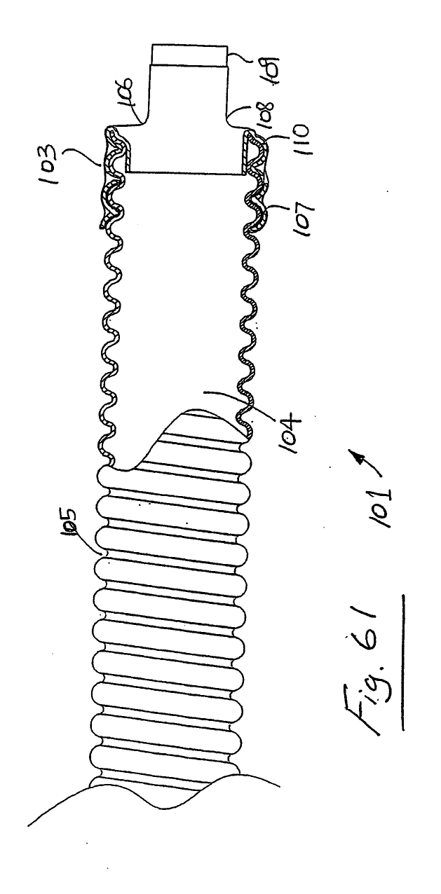

Manufacture of the overtube 101 will be described with reference to FIGS. 57 to 61. The overtube 101 is extruded to a typical length of 0.5 m with the convoluted corrugation 105 extending along the overtube 101 from the proximal end 102 to the distal end 103. The section of heat-shrink tubing 110 is positioned around the distal end 103 of the overtube 101, partially overlapping the distal end 103, and a mandrel 111 is partially inserted into the colonoscope lumen 104 from the distal end 103 (FIG. 57). Heat is applied to shrink the tubing 110 down partially onto the exterior surface of the overtube 101 and partially onto the mandrel 111. The mandrel 111 is moved further into the colonoscope lumen 104 while rotating the mandrel 111 (FIG. 58). By moving the mandrel 111 proximally, the tubing 110 is folded around the distal end 103 of the overtube 101 partially into the colonoscope lumen 104, and by rotating the mandrel 111, the tubing 110 is detached from the mandrel 111. The mandrel 111 is then removed from the colonoscope lumen 104.

A proximal end 112 of the tubular sheath 106 is rolled inwardly, and the sheath 106 is positioned around the distal end 103 of the overtube 101, partially overlapping the distal end 103. The tubular sheath 106 has a smaller diameter than the overtube 101, so the sheath 106 is stretched to position it around the distal end 103 of the overtube 101. The section of the heat-shrink tubing 107 is positioned around the sheath 106 distally of the rolled proximal end 112 (FIG. 59), and heat is applied to shrink the tubing 107 down onto the sheath 106 to fix the sheath 106 to the exterior surface of the overtube 101 (FIG. 60). The rolled proximal end 112 is then rolled out distally over the tubing 107, off the distal end 103 of the overtube 101 to define the outer sealing layer 108 around the inner sealing layer 109 (FIG. 61).

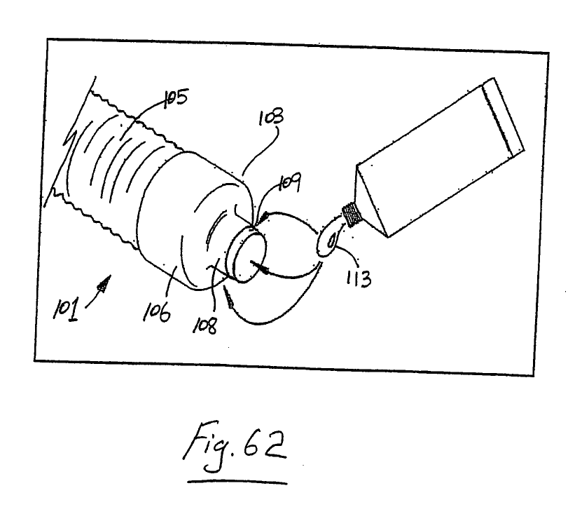

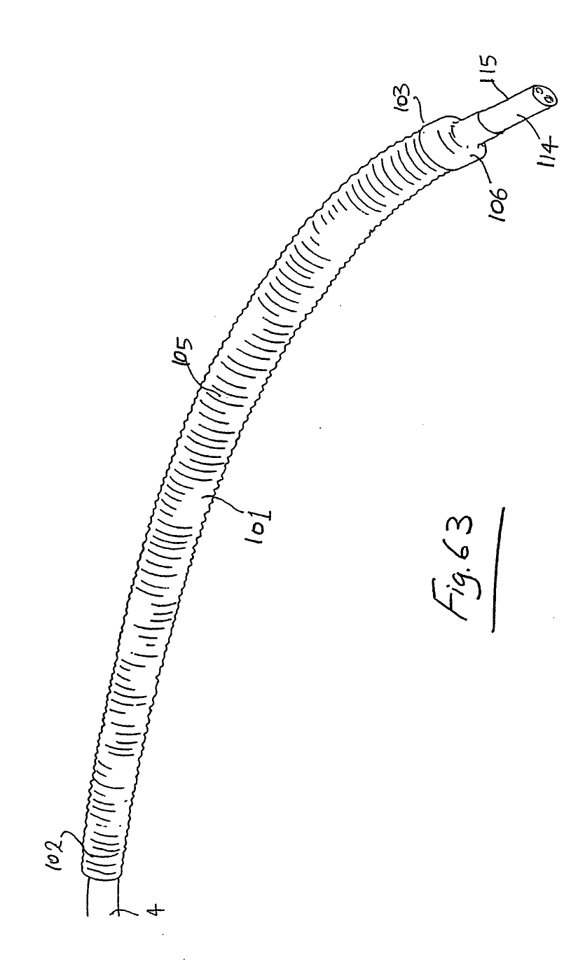

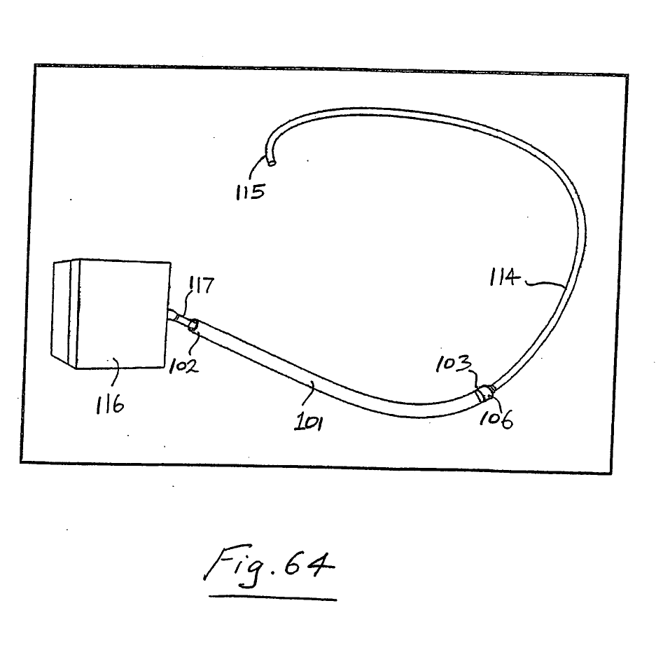

The assembled colonic overtube 101 is now ready for use. A biocompatible lubricant 113 is liberally applied both externally and internally to the overtube 101 (FIG. 62) to ease passage of the overtube 101 relative to a colonoscope and/or relative to a colon. A colonoscope 114 is inserted into the colonoscope lumen 104 at the proximal end 102 of the overtube 101 and advanced through the lumen 104 until a distal end 115 of the colonoscope 114 emerges from the distal end 103 of the overtube 101 through the sealing sheath 106 (FIG. 63).

The colonoscope 114 has a power/light source 116 at a proximal end 117 of the colonoscope 114, and the overtube 101 is moved proximally over the colonoscope 114 until the proximal end 102 of the overtube 101 is adjacent the power/light source 116 (FIG. 64).

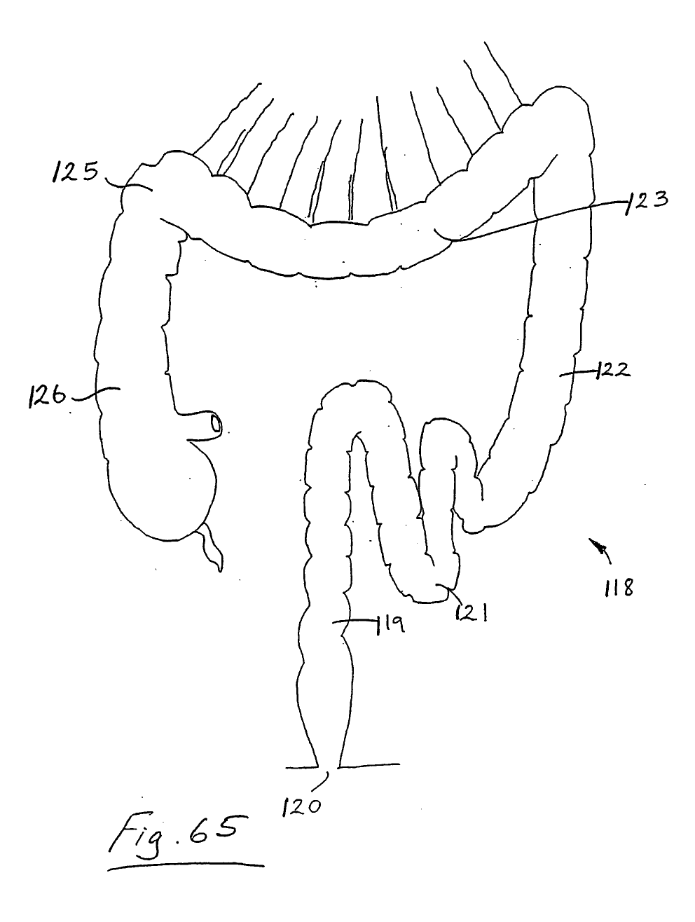

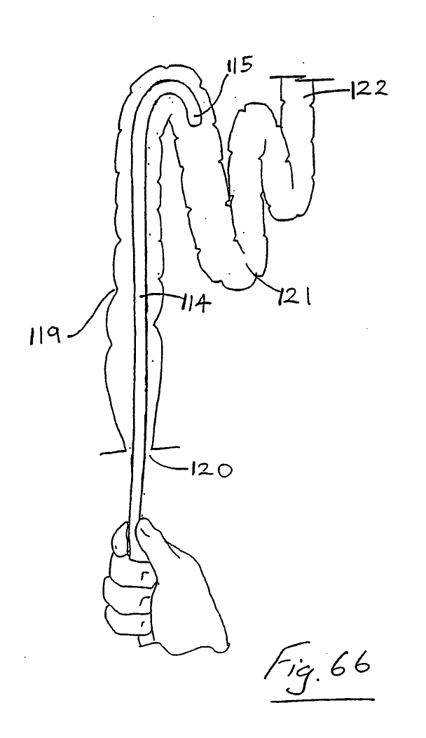

The colonoscope 114 is now ready for insertion into the colon of a patient. A typical colon 18 is illustrated in FIG. 65, in which the rectum 119 leads from the anus 120 to the sigmoid colon 121. The redundancy in the sigmoid colon 121 may be seen in FIG. 65. The descending colon 122 leads from the sigmoid colon 121 to the transverse colon 123, and the hepatic flexure 125 links the transverse colon 123 with the ascending colon 126.

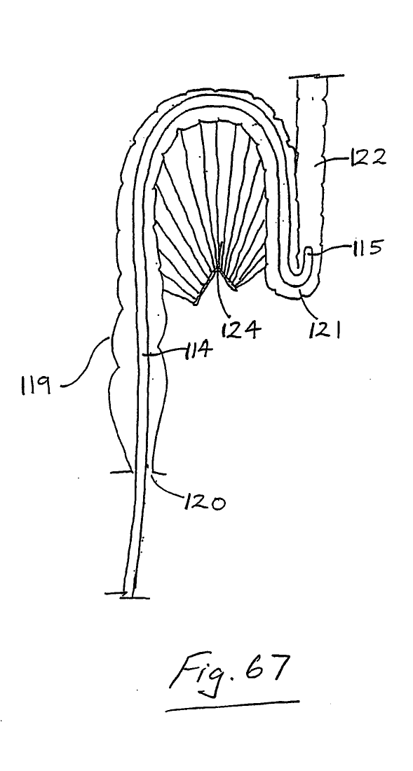

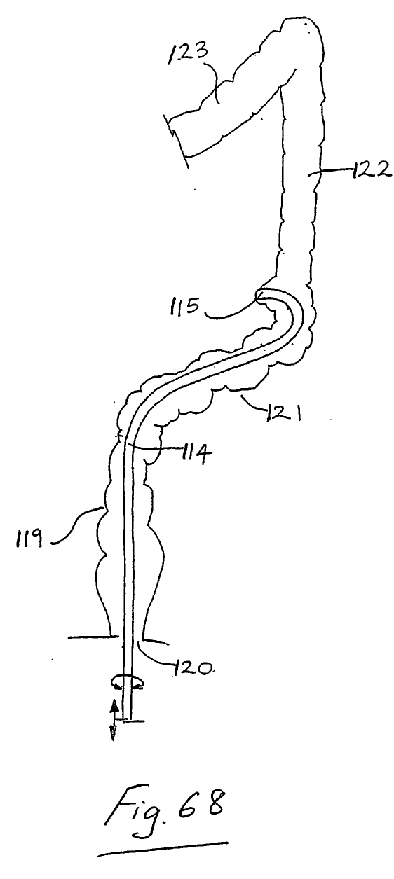

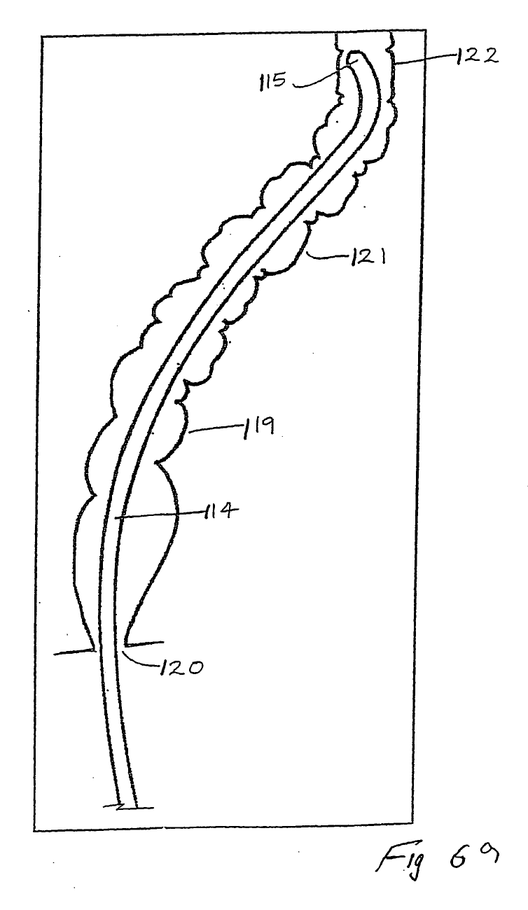

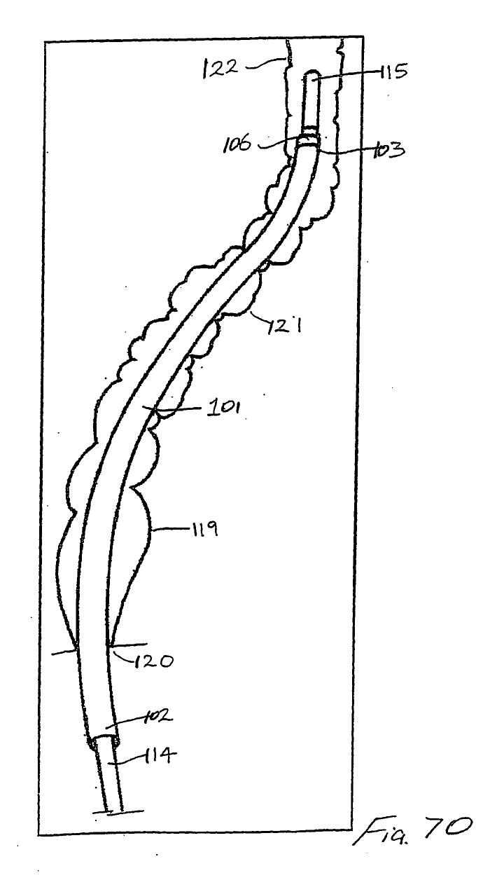

The distal end 115 of the colonoscope 114 is inserted through the anus 120 into the rectum 119, and the colonoscope 114 is advanced into the sigmoid colon 121 (FIG. 66). As the colonoscope 114 advances through the floppy sigmoid colon 121, a loop may form in the sigmoid colon 121, which results in stretching of the mesentery 124 to which the sigmoid colon 121 is attached (FIG. 67). When the distal end 115 of the colonoscope 114 reaches the proximal end of the descending colon 122, the distal end 115 is anchored in the fixed descending colon 122, and the sigmoid colon 121 is straightened by manipulating the colonoscope 114 (FIG. 68. When the sigmoid colon 121 has been straightened, the anchor is released (FIG. 69).

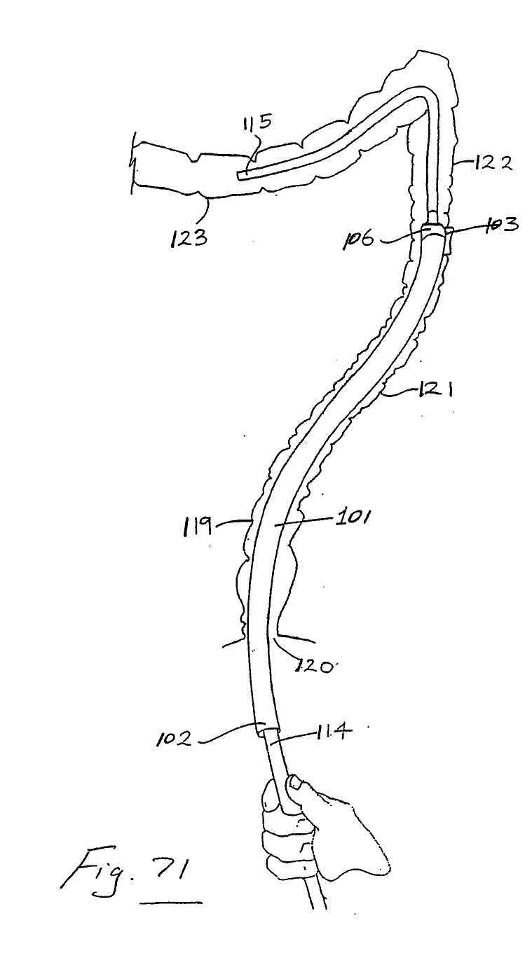

The distal end 103 of the overtube 101 is then inserted through the anus 120 into the rectum 119, and the overtube 101 is advanced through the straightened sigmoid colon 121 until the distal end 103 of the overtube 101 is at the proximal end of the descending colon 122 (FIG. 70). In this manner, the colonoscope 114 acts as a guiding track for the overtube 101 as it advances through the colon 118.

The sheath 106 effects a double-layered seal between the overtube 101 and the colonoscope 114 at the distal end 103 of the overtube 101. This seal ensures that no parts of the interior wall of the colon 118 become trapped between the colonoscope 114 and the overtube 101 as the overtube 101 is advanced over the colonoscope 114, and thus prevents shearing off of any parts of the colon wall, or puncturing the colon wall, or any other damage to the interior wall of the colon 118. The sealing sheath 106 also prevents faeces or other bodily materials from leaking between the colonoscope 114 and the overtube 101 proximally out through the anus 120.

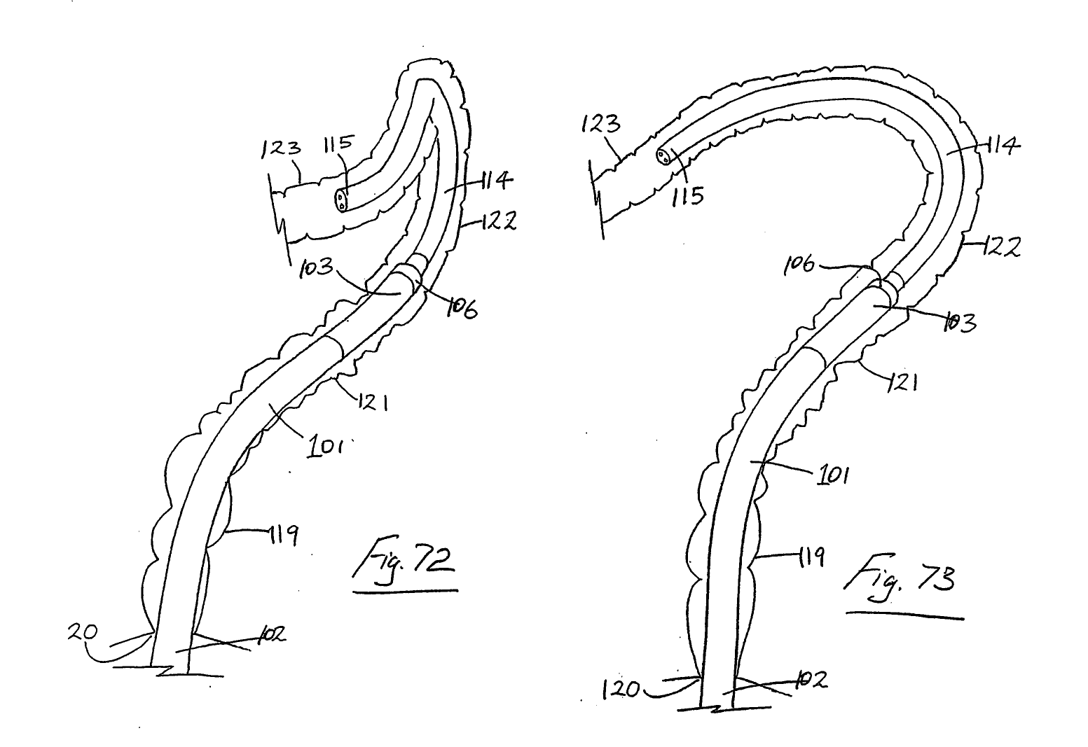

With the overtube 101 extended through the straightened sigmoid colon 121, as illustrated in FIG. 70, the colonoscope 114 may then be advanced further distally through the descending colon 122 and into the transverse colon 123 (FIG. 71). The overtube 101 acts as a splint to maintain the sigmoid colon 121 in the straightened configuration.

The splinting overtube 101 ensures that further advancement of the colonoscope 114 through the descending colon 122 and into the transverse colon 123 is possible by preventing loops from reforming in the sigmoid colon 121. In this manner, the overtube 101 minimises the pain or discomfort experienced by the patient during this procedure.

The overtube 101 may subsequently be further advanced through the colon 118 over the colonoscope 114 with the colonoscope 114 acting as a guiding track for the overtube 101.

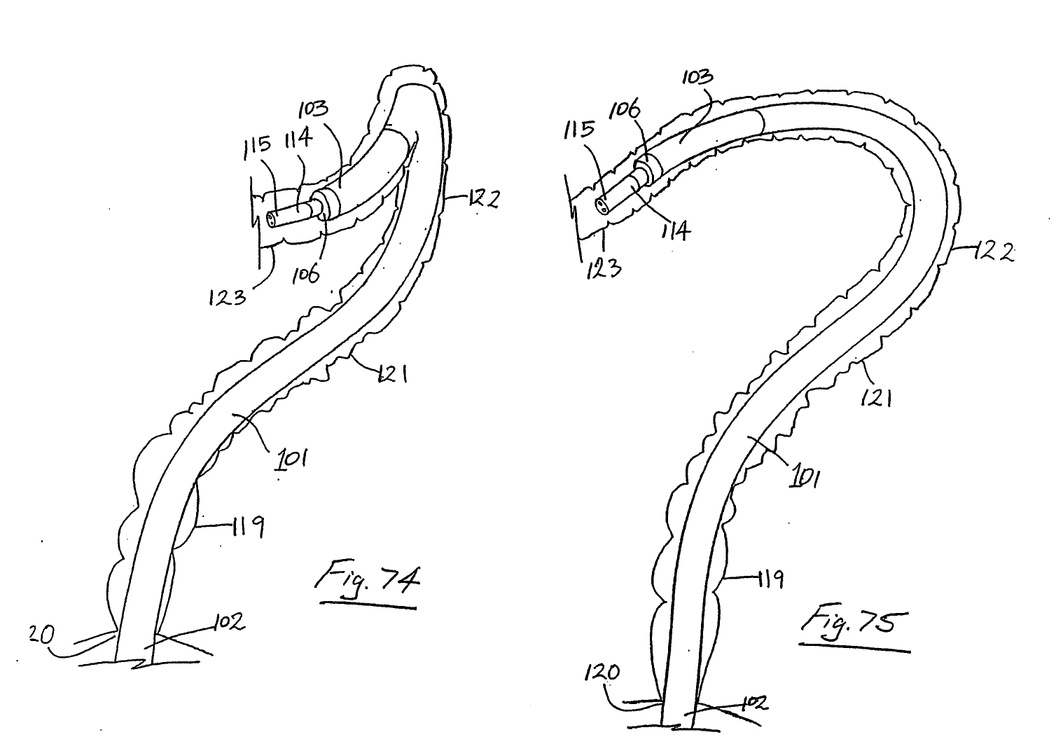

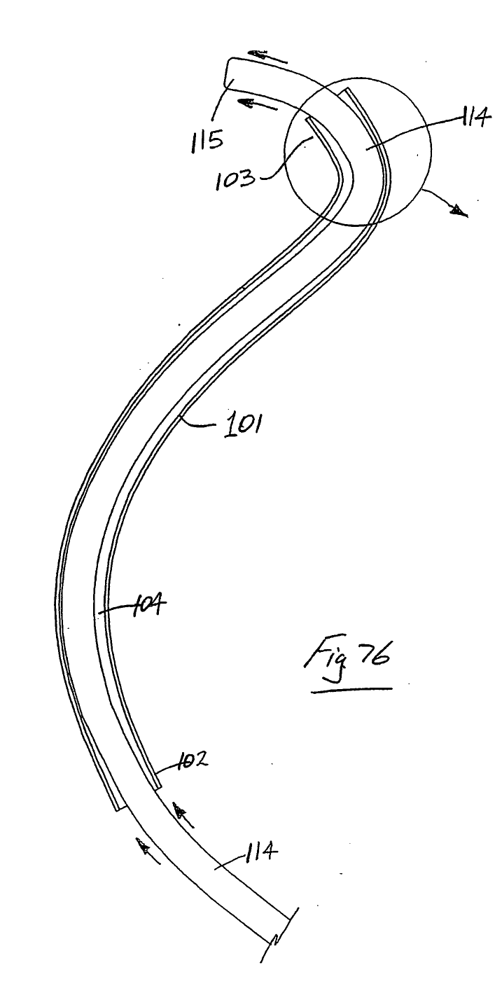

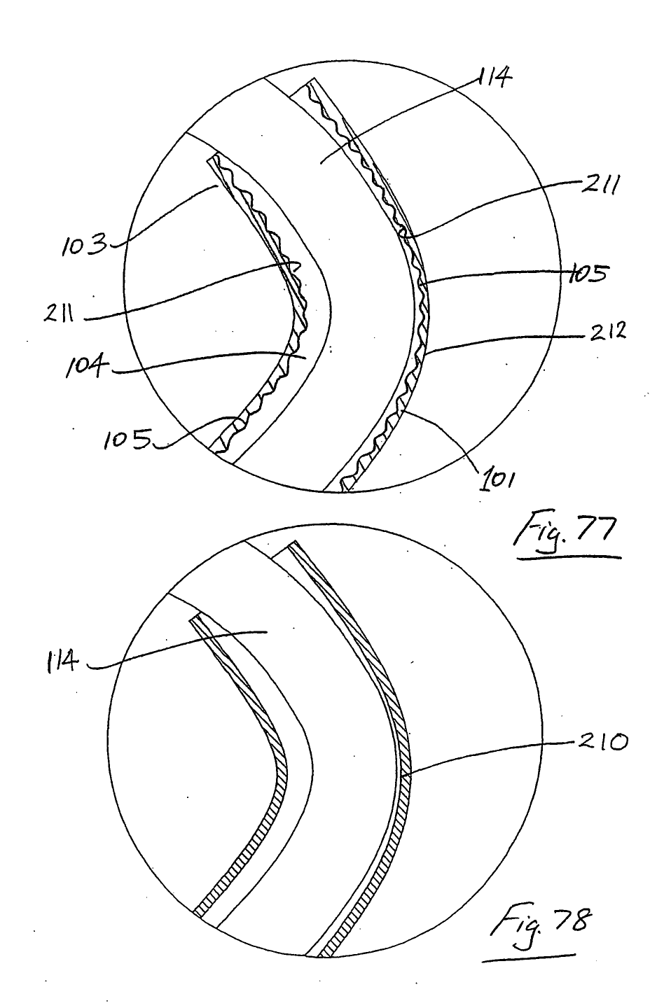

After the colonoscope 114 has been advanced from the descending colon 122 around the splenic flexure into the transverse colon 123, as illustrated in FIGS. 72 and 73, the overtube 101 may be advanced around the splenic flexure before advancing the colonoscope 114 further through the transverse colon 123, as illustrated in FIGS. 74 and 75.

FIG. 72 is a front view of the colon 118 and FIG. 73 is a side view of the colon 118 with the overtube 101 in the descending colon 122. FIG. 74 is a front view of the colon 118 and FIG. 75 is a side view of the colon 118 with the overtube 101 hooked around the splenic flexure.

It is believed that by hooking the overtube 101 around the splenic flexure before advancing the colonoscope 114 through the transverse colon 123, the subsequent advancement of the colonoscope 114 may be achieved while minimising stretching of the splenic flexure and/or the transverse colon 123, and also minimising the pain or discomfort experienced by the patient.

To more easily facilitate hooking of the overtube 101 around the relatively tight bend of the splenic flexure, the distal end 103 of the overtube 101 may be formed of a softer, more bendable material than the remainder of the overtube 101.

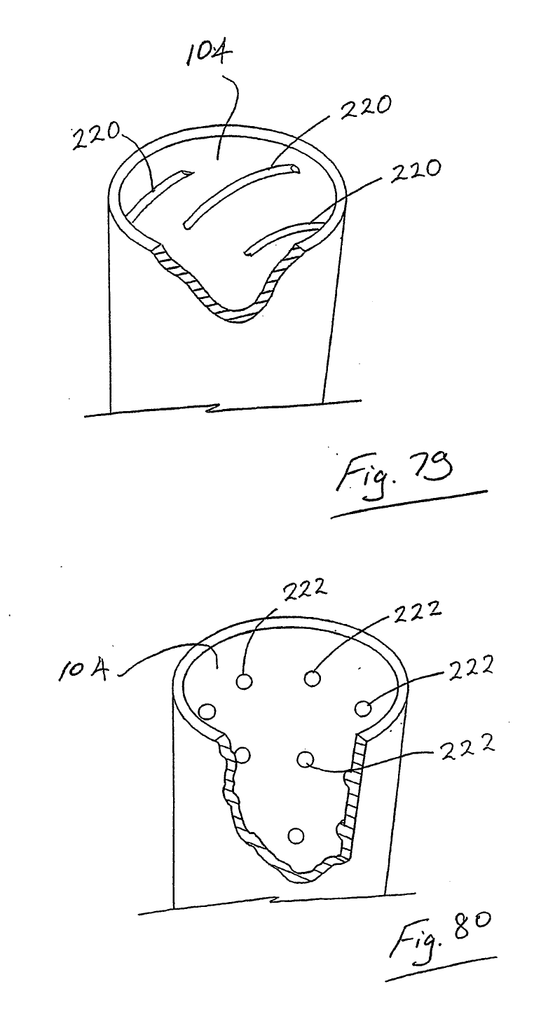

The corrugation 105 which extends along the overtube 101 in a convoluted manner results in a discontinuous interior surface 211 of the overtube 101, as illustrated in FIGS. 76 and 77. The corrugation 105 projects inwardly for contacting the colonoscope 114 in the colonoscope lumen 104. Thus, as the colonoscope 114 is advanced through the overtube 101, the area of contact between the colonoscope 114 and the corrugated overtube 101 is less than the area of contact that would otherwise result with a continuous interior surface 210, as illustrated in FIG. 78. Because the area of contact between the colonoscope 114 and the corrugated overtube 101 is reduced, the frictional force acting between the colonoscope 114 and the corrugated overtube 101 is also reduced. In this manner, the corrugated overtube 101 enables an easier passage of the colonoscope 114 through the colonoscope lumen 104 of the overtube 101.

The exterior surface 212 of the overtube 101 may be smooth, as illustrated in FIG. 77. This smooth surface 212 reduces the discomfort and/or pain experienced by the patient during the colonoscopy procedure while maintaining the kink-resistant and low-friction properties of the corrugation 105 on the interior surface 211.

It will be understood that the discontinuous nature of the interior surface of the overtube of the invention may be achieved in any suitable manner. For example, the overtube may comprise one or more inwardly projecting elements in the form of protruding strips 220, as illustrated in FIG. 79. The strips 220 may extend longitudinally along the overtube 101, or along the overtube 101 in a convoluted manner, or may extend at least partially circumferentially around the overtube 101. Alternatively the inwardly projecting elements may be provided in the form of a plurality of discrete protrusions 222, as illustrated in FIG. 80. By contacting a colonoscope in the colonoscope lumen 104, the inwardly projecting elements 220, 222 reduce the frictional force acting between the overtube 101 and the colonoscope, and thus ease passage of the overtube 101 over the colonoscope.

It will be appreciated that the corrugated overtube 101 may be provided in alternative forms to that described above. For example, the corrugation on the overtube 101 may extend at least partially circumferentially around the overtube 101, and/or more than one corrugation may be provided on the overtube 101.

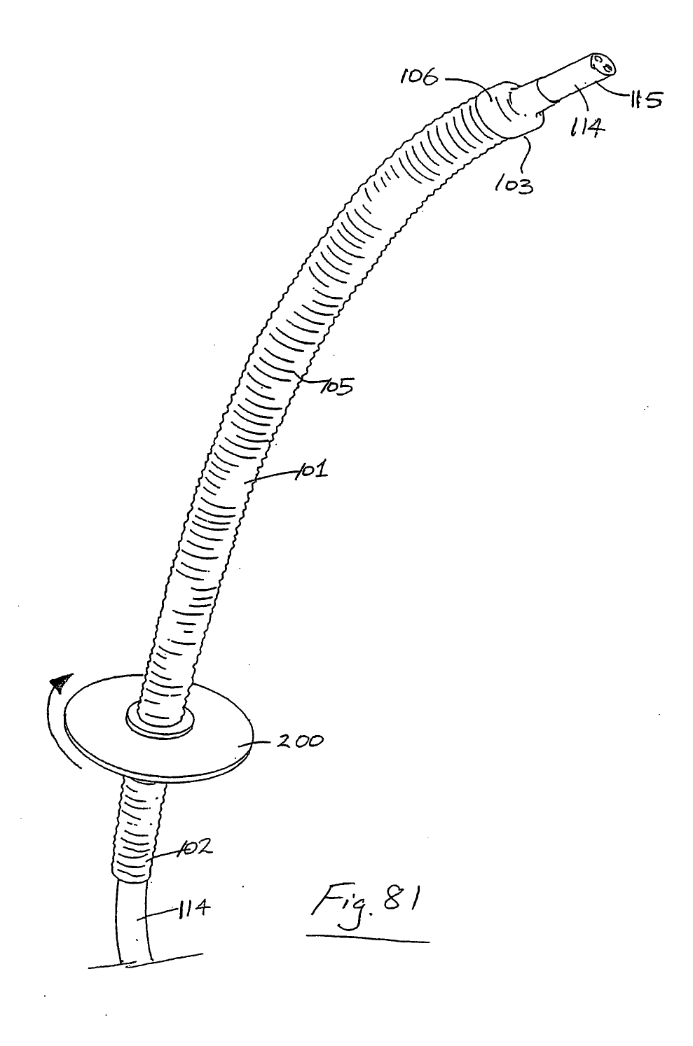

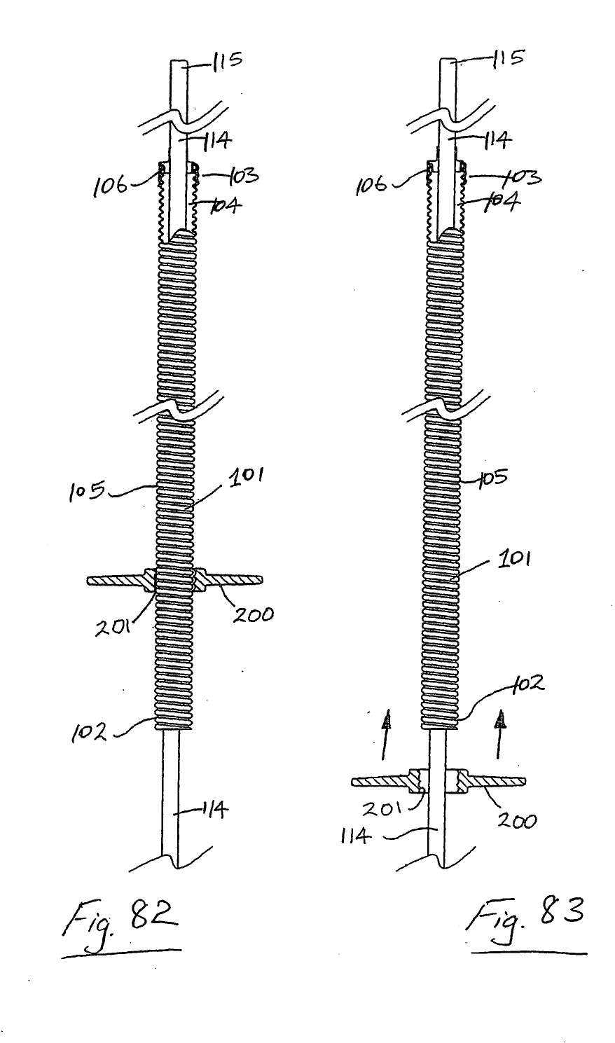

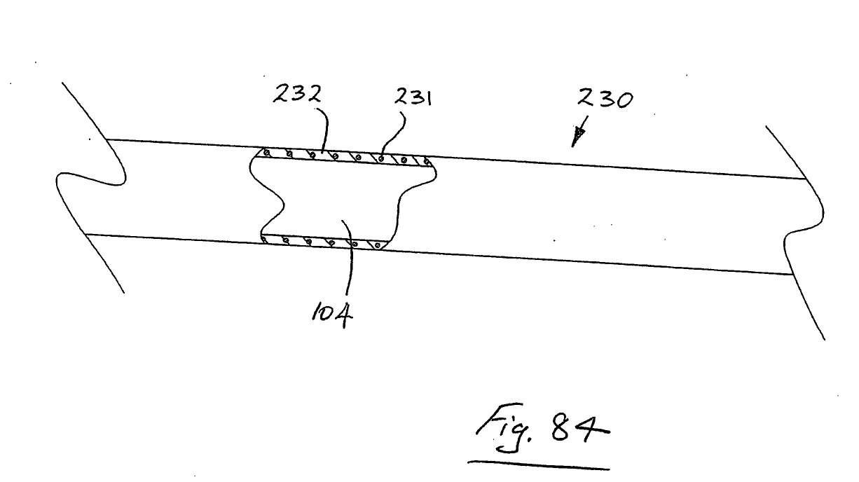

Referring to FIGS. 81 to 83, there is illustrated a flange 200 which may be used with the overtube 101 to prevent complete insertion of the overtube 101 into the colon 118. The flange 200 is releasably mounted to the overtube 101, in this case by means of a threaded arrangement 201.

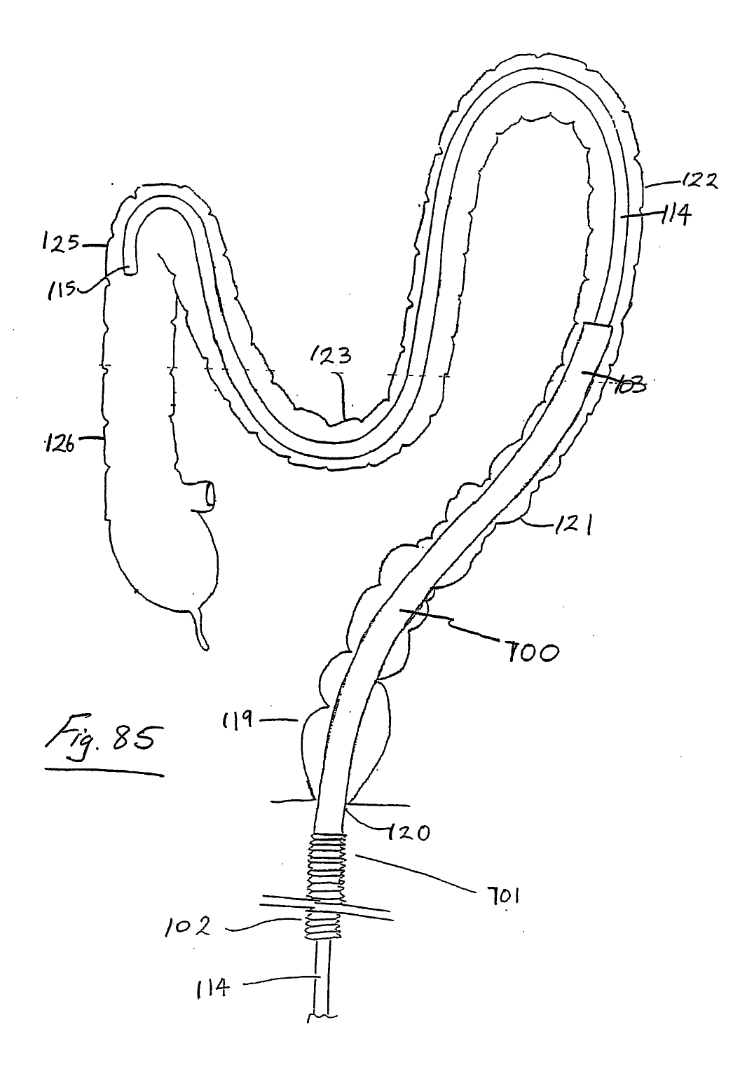

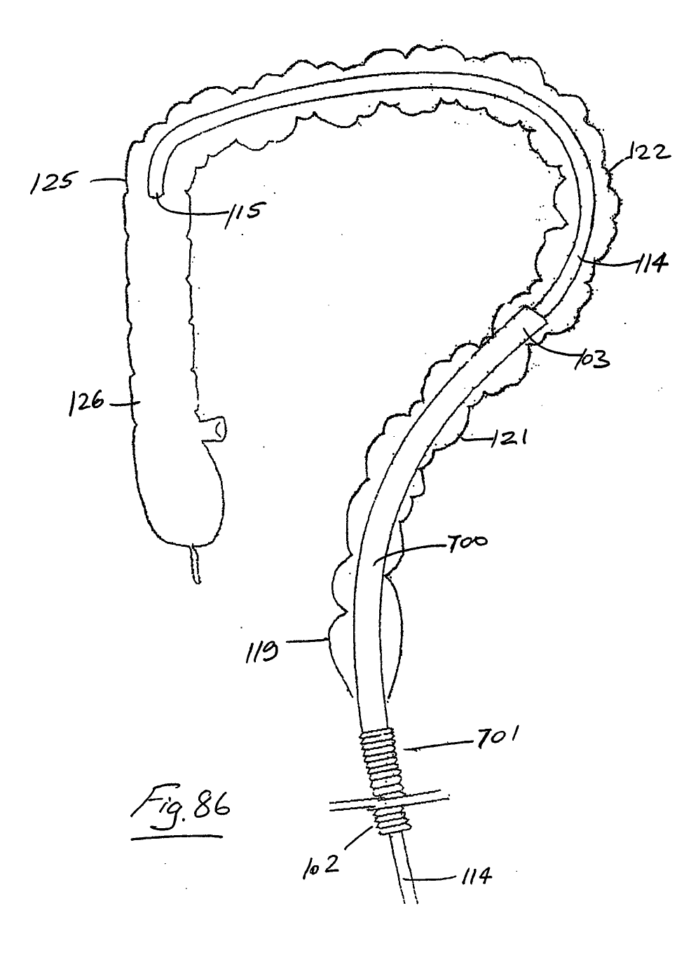

The threaded mounting arrangement enables the position of the flange 200 on the overtube 101 to be adjusted by a simple rotation of the flange 200 relative to the overtube 101, as illustrated in FIGS. 82 and 83. Because the flange position is adjustable the colonoscopist can quickly and effectively adjust the flange 200 to suit the particular characteristics of the colon 118 undergoing treatment.

It will be appreciated that the flange 200 may be provided with alternative means of adjusting the position on the overtube 101, and/or with alternative means of releasable mounting to the overtube 101. Also the flange 200 could alternatively be provided fixed to or integral with the overtube 101 towards the proximal end 102 of the overtube 101. Furthermore, the limiting means may be provided in an alternative form to a flange.

FIG. 84 illustrates another colonic overtube 230 according to the invention, which is similar to the overtube 101, and similar elements in FIG. 84 are assigned the same reference numerals. In this case, the overtube 230 comprises a reinforcement means, in the form of a coil 231 of metallic material embedded within the wall 232 of the overtube 230. This composite construction enables the overtube 230 to flex laterally during advancement over a colonoscope through a potentially tortuous path in a colon substantially without kinking.

It will be understood that the reinforcement means may be provided in any suitable form, such as a mesh, or a braided construction. In another alternative the composite overtube may have a layered construction.

It is to be understood that other configurations and constructions of overtube are also possible which are laterally flexible to facilitate flexing of the overtube substantially without kinking during advancement of the overtube through a colon.

More than one laterally flexible portion may be provided spaced along the overtube. The positioning and/or number of the laterally flexible portions may be selected to achieve the desired kink resistance.

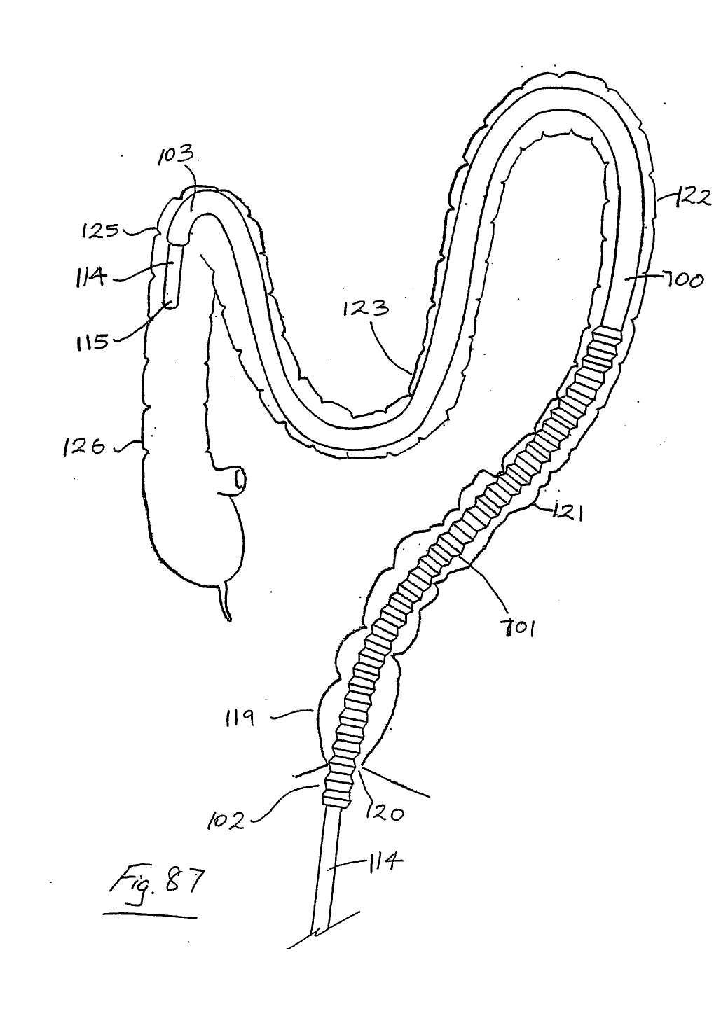

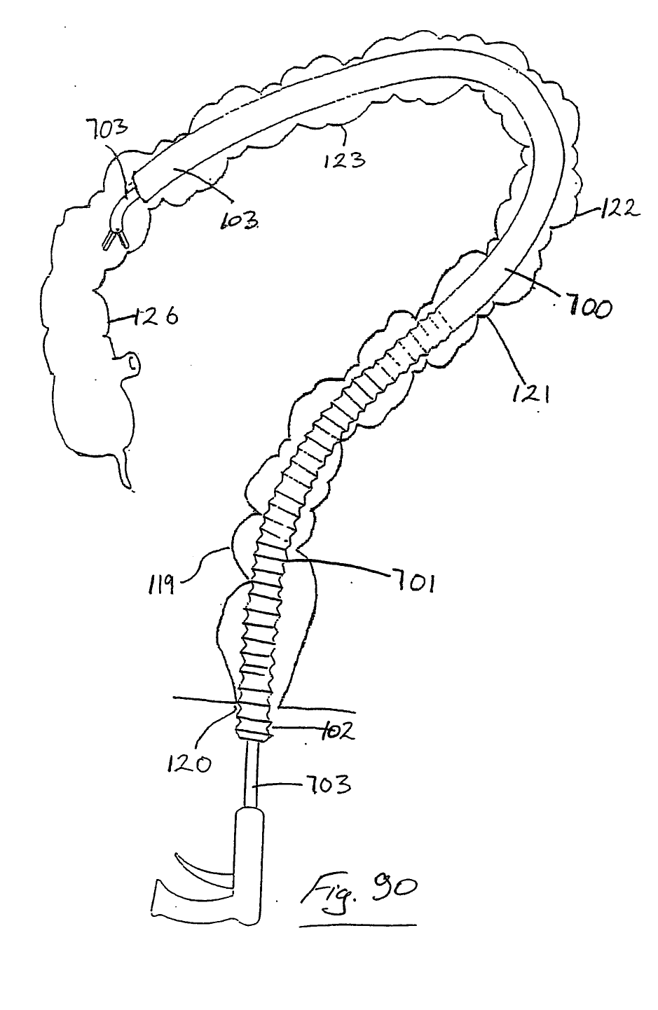

Referring to FIGS. 85 to 91, there is illustrated another colonic overtube 700 which is suitable for use in a method of performing a colonoscopy according to the invention. The overtube 700 may be used to cannulate a colon.

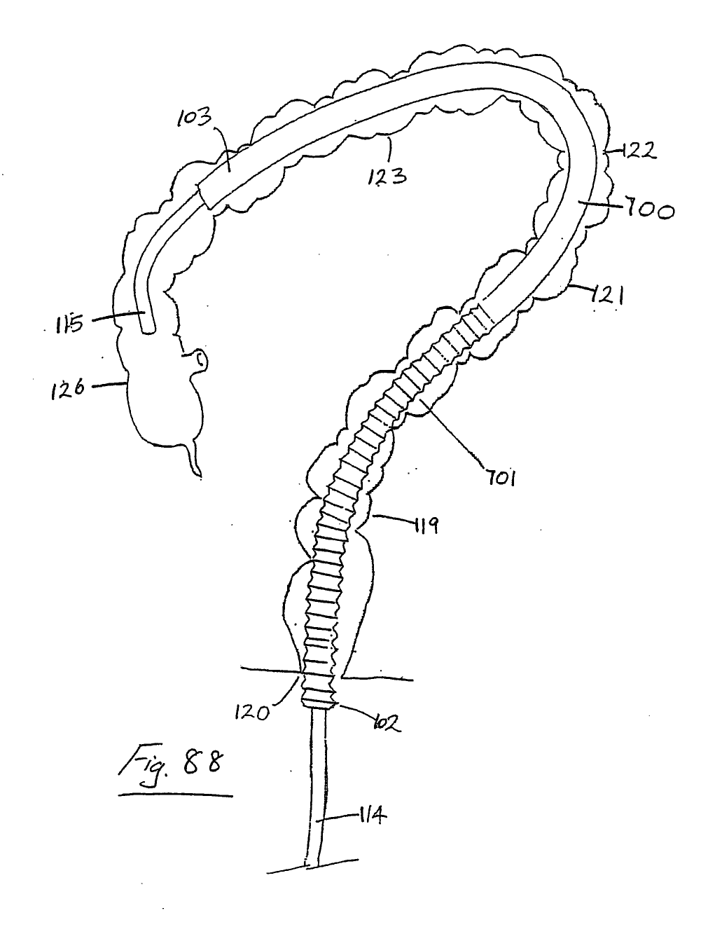

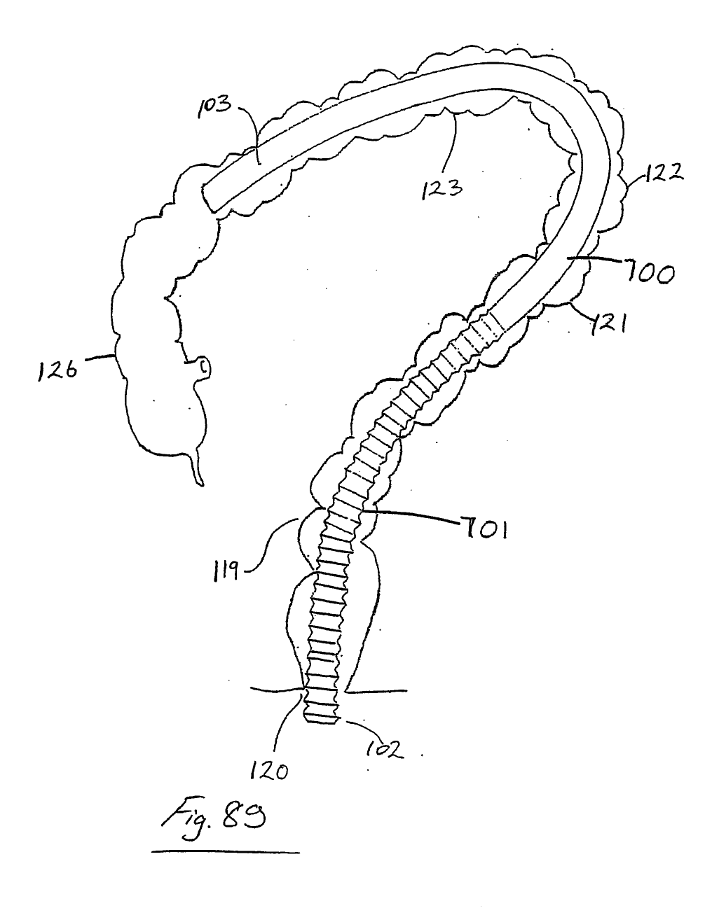

The overtube 700 is similar to the overtube 101 of FIGS. 55 to 77, and similar elements in FIGS. 85 to 91 are assigned the same reference numerals. The overtube 700 is extendable between a shortened configuration, as illustrated in FIG. 85, and an elongated configuration, as illustrated in FIGS. 86 to 91, for cannulating at least portion of the colon 118, in particular cannulating the colon 118 to a point distally of the descending colon 122. In this case, a portion 701 of the overtube 700 has a concertina-type configuration in the shortened configuration (FIG. 85), and an extended configuration in the elongated configuration (FIG. 87). The concertinaed portion 701 is provided at the proximal end 102 of the overtube 700.

In the method of performing a colonoscopy according to the invention, the overtube 700 is mounted to the colonoscope 114 with the portion 701 retracted into the concertina-like manner before insertion of the colonoscope 114 into the colon 118. Insertion of the colonoscope 114 into the colon 118, straightening of the sigmoid colon 121 and advancement of the overtube 700 over the colonoscope 114 are performed in a manner similar to that described previously with reference to FIGS. 66 to 71.

The overtube 700 acts as a splint to maintain the naturally floppy sigmoid colon 121 in the straightened configuration. The colonoscope 114 may therefore be easily advanced through the floppy transverse colon 123 to the hepatic flexure 125 (FIG. 85).

The floppy transverse colon 123 may now be reduced/straightened in either of two ways. The first is by manipulating the colonoscope 114 until the colon assumes the classic question mark “?” shape [FIG. 86]. The concertinaed portion 701 of the overtube 700 can now be extended from the shortened configuration to the elongated configuration by pushing the overtube distally from externally of the colon over the colonoscope 114 through the descending colon 122 and the reduced/straightened transverse colon until the distal end 103 of the overtube 700 reaches the hepatic flexure 125 (FIG. 87).

Alternatively, the concertinaed portion 701 of the overtube 700 may first be extended from the shortened configuration to the elongated configuration by pushing the overtube 700 distally from externally of the colon 118. In this way the overtube 700 is advanced over the colonoscope 114 through the descending colon 122 and the floppy transverse colon 123 until the distal end 103 of the overtube 700 reaches the hepatic flexure 125 (FIG. 87).

The floppy transverse colon 123 may then be straightened by manipulation of the colonscope 114 and/or the overtube 700 from externally of the colon 118 (FIG. 88).

The overtube 700 of the present invention acts as a colonic cannula and maintains, in a stable configuration, the sections of the colon 118 that are normally floppy and mobile such as the sigmoid colon 121 and the transverse colon 123. This gives the colon 118 the classic question mark configuration as shown in FIG. 86. The colonoscope 114 may therefore be advanced further into the ascending colon 126 to perform the desired colonoscopic procedure in a relatively easy, pain-free manner.

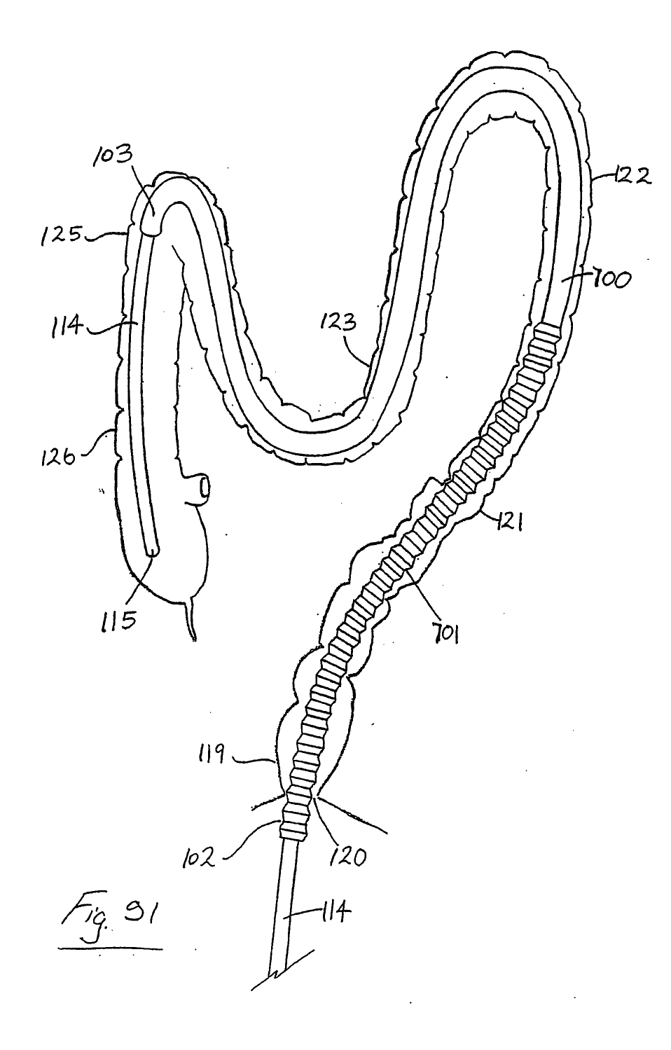

The colonoscope 114 may subsequently be removed through the colonoscope lumen 104 from the colon 118 leaving the overtube 700 in place in the cannulated colon 118 (FIG. 87). The overtube 700 can then be used to facilitate insertion of an endoscopic instrument through the overtube 700, for example an instrument 703 to remove polyps from the ascending colon 126 (FIG. 90), or the overtube 700 can be used to facilitate reinsertion of a colonoscope.

When the colonoscope 114 has been removed from the overtube 700, the overtube 700 provides a large working channel through the colon 118 through which any instrument may be quickly and easily passed to access any point in the colon 118 as far distally as the caecum. Rapid and less painful exchange of instruments and/or colonoscopes is thus facilitated by the overtube 700 because there is no contact between the instruments/colonoscopes and the inner wall of the colon 118 during insertion or withdrawal of the instruments/colonoscopes. In addition, the overtube 700 has a much larger diameter than the diameter of a typical colonoscope working channel. Thus, larger instruments may be used during a colonoscopy procedure with the overtube 700. Larger samples may also be removed using the overtube 700.

The colonoscope lumen 104 has a diameter, in this case approximately 15 mm, which results in a significantly larger cross sectional area than that of a typical colonoscope working channel.

If a subsequent region of interest in the colon 118 is proximally or distally of the distal end 103 of the overtube 700, the overtube 700 may be shortened or elongated until the distal end 103 is at the desired region of interest. Alternatively the overtube 700 may be withdrawn or advanced until the distal end 103 is at the desired region of interest. While shortening or withdrawal of the overtube 700 may be achieved by simply withdrawing the overtube 700 from the colon 118, advancement or lengthening of the overtube 700 is preferably achieved with the colonoscope 114 in situ in the colon 118.

The overtube 700 is removed from the colon 118 by collapsing the elongated portion 701 to the shortened configuration and withdrawing the overtube 700 proximally out of the colon 118. It is not necessary to reintroduce the colonoscope 114 into the colon 118 to facilitate removal of the overtube 700. Alternatively the overtube 700 may be withdrawn from the colon 118 leaving the colonoscope 114 in place in the colon 118. In this case, the colonoscope 114 may be subsequently withdrawn from the colon 118 thereby enabling the entire colon 118 to be examined during withdrawal of the colonoscope 114.

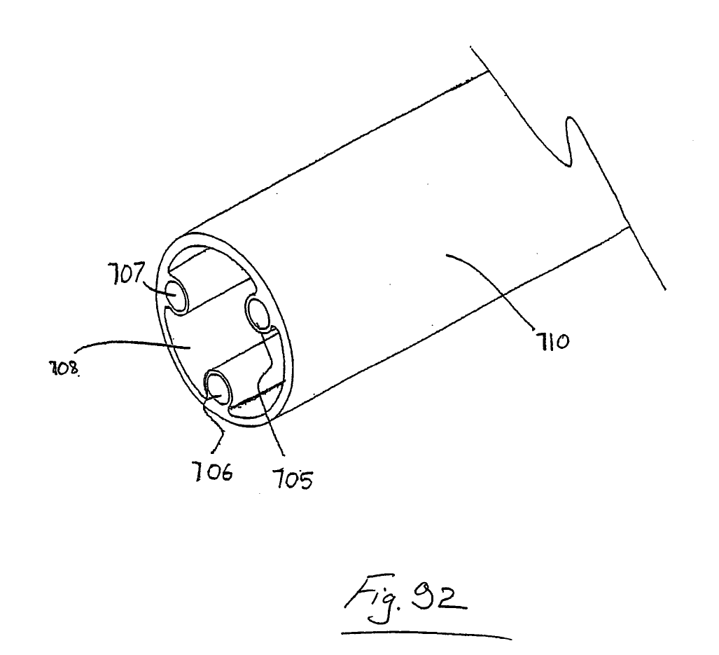

FIG. 91 illustrates an alternative method of performing a colonoscopy according to the invention, which is similar to the method described previously with reference to FIGS. 85 to 90. In this case, when the distal end 103 of the overtube 700 reaches the hepatic flexure 125, the transverse colon 123 is not straightened. Instead the colonoscope 114 is advanced further into the ascending colon 126, while the overtube 700 remains in position in the distended transverse colon 123 (FIG. 91).

In this manner, less time is required to perform the colonoscopy procedure because no straightening of the transverse colon 123 is required. In addition the pain or discomfort caused to the patient as a result of straightening of the transverse colon 123 is avoided using this method.

It will be appreciated that the overtube may be extended in a number of alternative ways. For example, the overtube may comprise a plurality of overtube sections which are releasably mountable to one another to extend the overtube to the elongated configuration in a manner similar to the extension of a chimney sweeping brush. As a further possibility the overtube may comprise one or more telescopable sections.

In an alternative arrangement, a connecting means, such as a drawstring, may be passed distally through the colonoscope working channel out of the distal end 115 of the colonoscope 114 and attached to the distal end 103 of the overtube 700. By maintaining the position of the colonoscope 114 fixed and pulling proximally on the connecting means from externally of the colon 118, the distal end 103 of the overtube 700 can be advanced over the colonoscope 102 thereby extending the concertinaed portion 701 of the overtube 700.

Other means of activating an actuator of the overtube from externally of the colon may also be applied to extend the overtube in situ to the elongated configuration. For example, the overtube may at least partially comprise an energy actuated polymer. By application of energy, such as a voltage difference across the overtube, a portion of the overtube may be extended.

The overtube 700 may have one or more laterally flexible portions spaced along the overtube 700, similar to the corrugated arrangement of FIG. 55, and/or the composite arrangement of FIG. 84. These laterally flexible portions may assist navigation of relatively tight bends in the colon 118, such as the splenic and hepatic flexures.

It is to be understood that a variety of different colonic overtubes may be employed in the method of performing a colonoscopy according to the invention, provided that the overtube may be advanced to cross the floppy transverse colon before further advancement of the colonoscope.

FIG. 92 illustrates another colonic overtube 710 according to the invention which is similar to the overtube 101 of FIGS. 55 to 77. The overtube 710 comprises at least one, and in this case three, exchange lumena 705, 706, 707, extending through the overtube 710 in addition to the colonoscope lumen 708. The exchange lumena 705, 706, 707 are suitable for exchanging a fluid, or a medical device through the lumena 705, 706, 707. For example, the lumen 705 may be used to provide a channel through which means for viewing the colon 118 from externally of the colon 118 can be provided, or the lumen 706 may be used to provide a channel through which means for illuminating the colon 118 can be provided.

It is highly advantageous to advance the overtube 710 with a visible path distally of the overtube 710 to ensure that no bowel is trapped at the distal end 103 of the overtube 710 during advancement through the colon 118.

As a further alternative, the lumen 707 may be used to provide a channel for flushing or insufflating the colon 118, for example to blow a protruding piece of the colon 118 laterally to clear a path for safe advancement of the overtube 710 through the colon 118.

In the case of the overtube 710 of FIG. 92, the exchange lumena 705, 706, 707 are provided on an interior surface of the overtube 710 extending inwardly into the colonoscope lumen 708. It will be appreciated that one or more of the exchange lumena may alternatively be provided on an exterior surface of the overtube 710 extending outwardly.

To assist with and speed up advancement of the colonic overtube into the colon 118 over the colonoscope 114 a guide device may be used, such as the guide device described in International Patent Application No. PCT/IE01/00039, the relevant contents of which are incorporated herein by reference.

The colonic overtube may be applied to maintain sections of the colon other than the sigmoid colon or the transverse colon in a straightened configuration. Indeed the overtube could also be applied to cannulate other body lumena, in which medical instruments are to be inserted.

The invention is not limited to the embodiments hereinbefore described, with reference to the accompanying drawings, which may be varied in construction and detail.

What is claimed is:

1. An evertable tube having a lumen therethrough, an inflation port for inflating the tube, and a self-closing valve at the inflation port.

2. A tube as claimed in claim 1 wherein the valve comprises a sheath extending from the port along a wall of the tube.

3. A tube as claimed in claim 2 wherein the sheath extends substantially parallel to a longitudinal axis of the tube.

4. A tube as claimed in claim 2 wherein the sheath is fixed to the wall of the tube.

5. A tube as claimed in claim 4 wherein the sheath is integral with the wall of the tube.

6. A tube as claimed in claim 2 wherein a sheath is of the same material as the wall of the tube.

7. A tube as claimed in claim 1 wherein the tube comprises at least two inflation ports.

8. A tube as claimed in claim 7 wherein the tube comprises a self-closing valve at each inflation port.

9. A tube as claimed in claim 1 wherein the tube is at least partially twisted.

10. A tube as claimed in claim 9 wherein the tube comprises means for adjusting the twist in the tube.

11. A tube as claimed in claim 9 wherein the tube comprises a substantially cylindrical outer sleeve section and a twisted inner sleeve section, the inner sleeve section being of a same untwisted diameter as that of the outer sleeve section.

12. A tube as claimed in claim 1 wherein at least a portion of the tube comprises a non-linear shape.

13. A tube as claimed in claim 12 wherein the tube is biased into the non-linear shape.

14. A tube as claimed in claim 12 wherein the tube is formed into the non-linear shape.

15. (canceled)

16. A guide device for a probe, the device comprising:
an evertable tube with a lumen therethrough, the tube being inflatable to grip a probe in the lumen such that the tube everts with advancement of the probe; and
means to for pulling the probe back relative to the tube to realign the probe relative to the tube.

17. A device as claimed in claim 16 wherein the device comprises means for longitudinally stiffening the tube.

18. A device as claimed in claim 17 wherein the stiffening means comprises at least one bracing column located within the tube.

19. A guide device for a probe, the device comprising:
an evertable tube with a lumen therethrough, the tube being inflatable to grip a probe in the lumen such that the tube everts with advancement of the probe; and
at least one stiffening column to facilitate moving at least part of the tube distally relative to a probe in the lumen and to align an end of the tube with an end of the probe.

20. A device as claimed in claim 18 wherein the column is tubular and extends co-axially around the lumen.

21. A device as claimed in claim 18 wherein the column comprises at least one corrugation for kink resistance.

22. A device as claimed in claim 21 wherein the corrugation extends along the column in a convoluted manner.

23. A device as claimed in claim 21 wherein the corrugation extends around the column in a loop.

24. A device as claimed in claim 19 wherein the at least one stiffening column comprises at least one slit through a column wall extending along the column in a spiral.

25. A device as claimed in claim 24 wherein an outer wall of the tube is connected at each end to an inner wall of the tube to define an enclosed inflation space therebetween.

26. A device as claimed in claim 16 wherein the tube comprises an evertable tube.

27. A device as claimed in claim 16 wherein the device comprises stopping means for selectively stopping tube eversion.

28. A device as claimed in claim 27 wherein the stopping means comprises a clamp engageable with the tube.

29. (canceled)

30. (canceled)

31. A probe assembly comprising:
a probe; and
a guide device for the probe, the guide device comprising:
an evertable tube with a lumen therethrough, the tube being inflatable to grip a probe in the lumen such that the tube everts with advancement of the probe.

32. An assembly as claimed in claim 31 wherein the probe comprises means for creating a fluid cushion between the probe and the guide device.

33. An assembly as claimed in claim 32 wherein the probe comprises one or more fluid openings on an outer surface of the probe for ejecting fluid therethrough to create the fluid cushion.

34. An assembly as claimed in claim 33 wherein the probe comprises a fluid inlet lumen in communication with the one or more fluid openings for passage of a fluid through the lumen and out through the one or more fluid openings.

35. An assembly as claimed in claim 32 wherein the probe comprises a fluid exhaust lumen for passage of a fluid through the lumen.

36. An assembly as claimed in claim 31 wherein the tube comprises an evertable tube.

37. An assembly as claimed in claim 31 wherein the device comprises a guide device.

38. An assembly as claimed in claim 31 wherein the probe comprises a colonoscope.

39. (canceled)

40. A device for straightening a looped section of a passageway, the device comprising:
an anchor for anchoring an interior wall of a passageway to the anchor; and
means for advancing the anchor through the passageway.

41. A device as claimed in claim 40 wherein at least part of the anchor is movable outwardly to anchor an interior wall of a passageway to the anchor by exerting outward pressure on the interior wall of the passageway.

42. A device as claimed in claim 41 wherein at least part of the anchor is inflatable.

43. A device as claimed in 40 wherein the anchor comprises an evertable tube.

44. (canceled)

45. A device as claimed in claim 43 wherein the anchor comprises a guide device.

46. A device as claimed in claim 40 wherein the means for advancing the anchor comprises a probe.

47. A device as claimed in claim 46 wherein the device comprises a probe assembly.

48. A device for straightening a sigmoid colon as claimed in claim 40.

49. (canceled)

50. A method of advancing a probe through a passageway, the method comprising:
providing a probe;
providing a guide device for the probe, the guide device having a lumen therethrough;
inserting the probe through the lumen of the guide device;
inflating the guide device to grip the probe;
advancing the probe through the passageway with an associated eversion of the guide device; and
advancing the probe relative to the guide device to align a leading end of the guide device with a leading end of the probe.

51. A method as claimed in claim 50 wherein the guide device is advanced over the probe by pushing the guide device over the probe.

52. A method as claimed in claim 50 wherein the method comprises the step of releasing a stop means before advancing at least part of the guide device over the probe.

53. A method as claimed in claim 50 wherein the probe is maintained in a fixed position during the step of advancing at least part of the guide device over the probe.

54. A method as claimed in claim 50 wherein the probe is advanced through the passageway by pushing the probe through the passageway.

55. A method of advancing a probe through a passageway, the method comprising:
providing a probe;
providing a guide device for the probe, the guide device having a lumen therethrough;
inserting the probe through the lumen of the guide device;
inflating the guide device to grip the probe;
pushing the probe to advance the probe through the passageway which causes eversion of the guide device; and
moving the probe relative to the guide device to align a leading end of the guide device with a leading end of the probe.

56. A method as claimed in claim 55 wherein at least part of the guide device is advanced over the probe to align the leading end of the guide device with the leading end of the probe.

57. A method as claimed in claim 55 wherein the probe is retracted through the lumen to align the leading end of the guide device with the leading end of the probe.

58. A method as claimed in claim 50 further comprising moving the guide device out of contact with the probe before retracting the probe relative to the guide device.

59. A method of advancing a probe through a passageway, the method comprising:
providing a probe;
providing a guide device for the probe, the guide device having a lumen therethrough;
inserting the probe through the lumen of the guide device;
inflating the guide device to grip the probe;
advancing the probe through the passageway with an associated eversion of the guide device;
moving the guide device out of contact with the probe; and
moving at least part of the guide device distally relative to the probe to align a leading end of the guide device with a leading end of the probe.

60. A method as claimed in claim 59 wherein the guide device is moved out of contact with the probe by deflating the guide device.

61. A method as claimed in claim 59 wherein the guide device is moved out of contact with the probe by creating a fluid cushion between the guide device and the probe.

62. A method as claimed in claim 50 releasing a stop means before eversion of the guide device.

63. A method as claimed in 50 wherein at least some of the steps are repeated to advance the probe in an incremental manner through the passageway.

64. A method of advancing a colonoscope through a colon as claimed in claim 50.

65. (canceled)

66. A method of straightening a looped section of a passageway, the method comprising:
providing an anchoring device;
advancing the anchoring device through the passageway;
anchoring an interior wall of the passageway to the anchoring device; and
moving the anchoring device proximally to at least partially straighten the looped section of the passageway.

67. A method as claimed in claim 66 wherein the anchoring device exerts outward pressure on the interior wall of the passageway to anchor the interior wall of the passageway to the anchoring device.

68. A method as claimed in claim 66 wherein the interior wall of the passageway is anchored to the anchoring device during advancement of the anchoring device through the passageway.

69. A method as claimed in claim 66 wherein the anchoring device everts during advancement through the passageway.

70. A method as claimed in claim 66 wherein at least some of the steps are repeated to straighten the looped section of the passageway in an incremental manner.

71. (canceled)

72. A method of straightening a sigmoid colon as claimed in claim 66.

73. (canceled)

74. A method of performing a colonoscopy procedure, the method comprising:
inserting a colonoscope into a colon;
inserting a colonic overtube into the colon;
advancing the colonoscope through a floppy section of the colon;
straightening the floppy section of the colon;
advancing the colonic overtube over the colonoscope through the floppy section of the colon; and
advancing the colonoscope distally of the straightened section of the colon.

75. A method as claimed in claim 74 wherein the floppy section of the colon is straightened by manipulating at least one of the colonoscope and the colonic overtube from externally of the colon.

76. A method as claimed in claim 74 wherein the colonic overtube is advanced distally of the straightened section of the colon.

77. A method of performing a colonoscopy procedure, the method comprising:
inserting a colonoscope into a colon;
inserting a colonic overtube into the colon;
advancing the colonoscope through a floppy section of the colon;
advancing the colonic overtube over the colonoscope through the floppy section of the colon; and
advancing the colonoscope distally of the floppy section of the colon.

78. A method as claimed in claim 77 wherein the colonic overtube is advanced distally of the floppy section of the colon.

79. A method as claimed in claim 74 wherein the floppy section of the colon comprises the transverse colon.

80. A method of performing a colonoscopy procedure, the method comprising:
inserting a colonoscope into a colon;
inserting a colonic overtube into the colon;
advancing the colonoscope around a bend in the colon;
advancing the colonic overtube over the colonoscope around the bend in the colon; and
advancing the colonoscope distally of the bend in the colon.

81. A method as claimed in claim 80 wherein the colonic overtube remains in a fixed position during advancement of the colonoscope distally of the bend in the colon.

82. A method as claimed in claim 80 wherein the bend comprises the splenic flexure.

83. A method as claimed in claim 74 further comprising withdrawing the colonoscope from the colon while the colonic overtube remains in place in the colon.

84. A method as claimed in claim 83 further comprising:
advancing a medical device through the colonic overtube to access a point in the colon distally of the colonic overtube.

85. A method as claimed in claim 74 further comprising:
mounting the colonic overtube to the colonoscope before inserting the colonoscope into the colon.

86. A method as claimed in claim 74 wherein the colonic overtube is advanced by extending at least part of the colonic overtube from a shortened configuration to an elongated configuration.

87. A method as claimed in claim 74 wherein the colonic overtube is advanced by pushing the colonic overtube from externally of the colon.

88. (canceled)
